(19) United States
(12) Patent Application Publication
(10) Pub. No.: US 2026/0308175 A1
(43) Pub. Date: Oct. 8, 2026

(54) IMIDAZOLIUM SALT MACROCYCLIC COMPOUND AND PREPARATION METHOD THEREFOR AND USE THEREOF

(71) Applicant: FUDAN UNIVERSITY, Shanghai (CN)

(72) Inventors: Zhanting LI, Shanghai (CN); Danwei ZHANG, Shanghai (CN); Wei ZHOU, Shanghai (CN); Jingyu YANG, Shanghai (CN); Yan WU, Shanghai (CN); Yueyang LIU, Shanghai (CN); Hui WANG, Shanghai (CN)

(21) Appl. No.: 19/132,964

(22) PCT Filed: Jul. 8, 2024
(86) PCT No.: PCT/CN2024/104296

§ 371 Date: May 27, 2025

(30) Foreign Application Priority Data
Jul. 17, 2023 (CN) 202310875059.4

(51) Int. Cl. C07D 498/22; A61K 31/4188; A61P 21/02

(52) U.S. Cl. CPC C07D 498/22; A61K 31/4188; A61P 21/02

(57) ABSTRACT

An imidazolium salt macrocyclic compound, and a preparation method therefor, and a use thereof are provided. The macrocyclic compound as shown in formula (I) or a pharmaceutically acceptable salt, a hydrate, or a solvate thereof can be used in a composition with a pharmaceutically acceptable carrier, which is capable of producing neuromuscular junction block in the preparation of muscle relaxants.

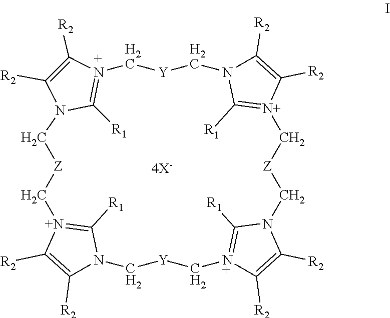

### TECHNICAL FIELD

The present invention belongs to the field of medicine, specifically, the present invention relates to an imidazolium salt macrocyclic compound and preparation method therefor and use thereof as muscle relaxants.

### BACKGROUND

Neuromuscular blocking agents (also known as muscle relaxants) are widely used in clinical anesthesia surgery to relax skeletal muscles, achieve rapid intubation, and provide safe surgical conditions. Every year, hundreds of millions of patients worldwide receive muscle relaxants during surgery. Among them, non-depolarizing muscle relaxants such as rocuronium bromide, cisatracurium besylate, vecuronium bromide, and pancuronium bromide have fewer side effects and longer effective times than depolarizing muscle relaxants, and are therefore more widely used in clinical practice, accounting for over 95% of clinical use.

Non-depolarizing muscle relaxants with moderate efficacy, such as rocuronium bromide, vecuronium bromide, and cisatracurium besylate, can be administered to adult patients at twice the ED95 dose, with an effective muscle relaxation time of 30-45 minutes. Injecting twice the ED95 dose of long-acting muscle relaxant pancuronium bromide can maintain effective muscle relaxation for approximately 75 minutes in adult patients. For general anesthesia surgery with a duration exceeding the effective muscle relaxation time mentioned above, it is necessary to administer single or multiple additional injections or continuous injections of muscle relaxants to the patient, which increases the cost of medication in different degrees. In addition, single or multiple additional injections of muscle relaxants require the determination of the appropriate injection dose and injection time, but it may still lead to fluctuations or abnormalities in muscle relaxation. Continuous injection also requires the determination of the appropriate maintenance dose. Both methods depend highly on multiple types of medical equipment monitoring, rely on the experience and professional skills of anesthesiologists and nurses, which increase or prolong the workload of anesthesiologists and nurses, and generate higher surgical costs overall.

In theory, if a muscle relaxant can produce sufficient and effective muscle relaxation time, it is possible to achieve the injection of muscle relaxant once per surgery, avoiding the aforementioned intermittent or continuous additional administration and the resulting increase in overall economic costs. On the premise of ensuring safety, achieving rapid onset and rapid antagonism after surgery would significantly reduce the complexity of muscle relaxant administration during surgical anesthesia, enhance surgical efficiency and medical resource utilization, and ultimately reduce lower surgical costs, thereby benefiting patients. The long-acting muscle relaxant currently used in clinical practice, pancuronium bromide, has a relatively short duration of action and cannot meet the requirements of single administration for surgeries lasting more than 75 minutes, and also has multiple side effects and contraindications, and there is no rapid antagonist that specifically binds to it. Therefore, there is currently no drug combination that achieves long-term muscle relaxation and rapid antagonism in clinical practice. It is a highly challenging work to develop a drug combination that meets the requirements of the abovementioned performance.

The muscle relaxants currently used in clinical practice are different types of tetraalkyl cationic ammonium salt derivatives. Long-term acting muscle relaxants generally have a slower onset of action, and its rapid onset can be achieved through two pathways. One is to increase the dosage of muscle relaxants. This method of administration is the simplest and does not require combination with other muscle relaxants, avoiding possible side effects caused by muscle relaxant interactions. However, muscle relaxants require a wider dosage window. The other is to use it in combination with rapid-acting muscle relaxants currently used in clinical practice such as rocuronium bromide. This strategy does not require an increase in the dosage of long-acting muscle relaxants, and this combination therapy strategy of muscle relaxants with different pharmacological effects has been widely used in clinical practice. If safe, rapid-acting, and long-acting muscle relaxants can be obtained, specific antagonists can be designed based on their structural characteristics to achieve clinical rapid antagonism. As the key first step in developing a new combination drug of long-acting muscle relaxants and rapid antagonists, it is necessary to firstly develop safe muscle relaxants that can act rapidly and have a long acting duration, providing candidate structures for further development of specific rapid antagonists.

Therefore, there is an urgent need in this field to develop a muscle relaxant that can achieve rapid onset, long-term efficacy, and high safety to meet the needs of clinical use.

### SUMMARY OF THE INVENTION

One purpose of the present invention is to provide a muscle relaxant with rapid onset and long acting duration.

Another purpose of the present invention is to provide a pharmaceutical combination of a muscle relaxant and a muscle relaxant antagonist.

In the first aspect of the present invention, provided is an imidazolium salt macrocyclic compound of formula I, or a pharmaceutically acceptable salt, a hydrate, or a solvate thereof,

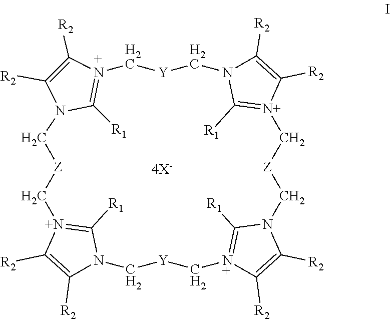

each R₁ is independently selected from the group consisting of: H, D, substituted or unsubstituted C₁₋₂₀ alkyl, substituted or unsubstituted 1-20 membered heteroalkyl containing 1 to 6 heteroatoms selected from nitrogen, oxygen, and sulfur, substituted or unsubstituted C₂₋₂₀ alkenyl, substituted or unsubstituted 2-20 membered heteroalkenyl containing 1 to 3 heteroatoms selected from nitrogen, oxygen, and sulfur, substituted or unsubstituted C₂₋₂₀ alkynyl, substituted or unsubstituted 2-20 membered heteroalkynyl containing 1 to 3 heteroatoms selected from nitrogen, oxygen, and sulfur, substituted or unsubstituted C₃₋₂₀ cycloalkyl, substituted or unsubstituted 3-20 membered heterocyclyl containing 1 to 6 heteroatoms selected from nitrogen, oxygen, and sulfur;
each R₂ is independently selected from the group consisting of: H, substituted or unsubstituted C₁₋₂₀ alkyl, substituted or unsubstituted 1-20 membered heteroalkyl containing 1 to 6 heteroatoms selected from nitrogen, oxygen, and sulfur, substituted or unsubstituted C₂₋₂₀ alkenyl, substituted or unsubstituted 2-20 membered heteroalkenyl containing 1 to 3 heteroatoms selected from nitrogen, oxygen, and sulfur, substituted or unsubstituted C₂₋₂₀ alkynyl, substituted or unsubstituted 2-20 membered heteroalkynyl containing 1 to 3 heteroatoms selected from nitrogen, oxygen, and sulfur, substituted or unsubstituted C₃₋₂₀ cycloalkyl, substituted or unsubstituted 3-20 membered heterocyclyl containing 1 to 6 heteroatoms selected from nitrogen, oxygen, and sulfur, or two adjacent R₂ together with their attached carbon atoms form a group selected from the group consisting of: substituted or unsubstituted C₃₋₂₀ cycloalkyl, substituted or unsubstituted C₆₋₂₀ aryl, substituted or unsubstituted 3-20 membered heterocyclyl containing 1 to 6 heteroatoms selected from nitrogen, oxygen, and sulfur, and substituted or unsubstituted 5-20 membered heteroaryl containing 1 to 6 heteroatoms selected from nitrogen, oxygen, and sulfur;
each Y and Z is independently selected from the group consisting of: null, substituted or unsubstituted C₁₋₂₀ alkylene, substituted or unsubstituted C₁₋₂₀ alkylene containing 1 to 6 heteroatoms selected from nitrogen, oxygen, and sulfur, substituted or unsubstituted C₃₋₂₀ cycloalkylene, substituted or unsubstituted C₅₋₂₀ arylene, substituted or unsubstituted Ar1-L-Ar2, and substituted or unsubstituted 5-20 membered heteroarylene;
L is selected from the group consisting of: null, O, NH, C₁₋₁₀ alkylene, S, and phenylene;
Ar1 and Ar2 are each independently null, C₆₋₁₀ arylene, or 5-8 membered heteroarylene;
X is a pharmaceutically acceptable anion charge-balanced with the tetravalent cationic imidazolium macrocycle in the structure of formula I;
unless otherwise specified, the “substituted” refers to the substitution of one or more hydrogens on a group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, each R₁ is independently selected from the group consisting of: H, D, substituted or unsubstituted C₁₋₁₀ alkyl, substituted or unsubstituted 1-10 membered heteroalkyl containing 1 to 6 heteroatoms selected from nitrogen, oxygen, and sulfur, substituted or unsubstituted C₂₋₁₀ alkenyl, substituted or unsubstituted 2-10 membered heteroalkenyl containing 1 to 3 heteroatoms selected from nitrogen, oxygen, and sulfur, substituted or unsubstituted C₂₋₁₀ alkynyl, substituted or unsubstituted 2-10 membered heteroalkynyl containing 1 to 3 heteroatoms selected from nitrogen, oxygen, and sulfur, substituted or unsubstituted C₃₋₁₀ cycloalkyl, and substituted or unsubstituted 3-10 membered heterocyclyl containing 1 to 6 heteroatoms selected from nitrogen, oxygen, and sulfur;
wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, each R₁ is independently selected from the group consisting of: H, D, substituted or unsubstituted C₁₋₆ alkyl, substituted or unsubstituted 1-6 membered heteroalkyl containing 1 to 3 heteroatoms selected from nitrogen, oxygen, and sulfur, substituted or unsubstituted C₂₋₆alkenyl, substituted or unsubstituted 2-6 membered heteroalkenyl containing 1 to 3 heteroatoms selected from nitrogen, oxygen, and sulfur, substituted or unsubstituted C₂₋₆ alkynyl, substituted or unsubstituted 2-6 membered heteroalkynyl containing 1 to 3 heteroatoms selected from nitrogen, oxygen, and sulfur, substituted or unsubstituted C₃₋₇ cycloalkyl, substituted or unsubstituted 3-7 membered heterocyclyl containing 1 to 3 heteroatoms selected from nitrogen, oxygen, and sulfur;
wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, each R₁ is independently selected from the group consisting of: H, D, substituted or unsubstituted C₁₋₆ alkyl, substituted or unsubstituted 1-6 membered heteroalkyl containing 1 to 3 heteroatoms selected from nitrogen, oxygen, and sulfur, substituted or unsubstituted C₂₋₆alkenyl, and substituted or unsubstituted C₃₋₇ cycloalkyl;
wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, each R₂ is independently selected from the group consisting of: H, substituted or unsubstituted C₁₋₁₀ alkyl, substituted or unsubstituted 1-10 membered heteroalkyl containing 1 to 6 heteroatoms selected from nitrogen, oxygen, and sulfur, substituted or unsubstituted C₂₋₁₀ alkenyl, substituted or unsubstituted 2-10 membered heteroalkenyl containing 1 to 3 heteroatoms selected from nitrogen, oxygen, and sulfur, substituted or unsubstituted C₂₋₁₀ alkynyl, substituted or unsubstituted 2-10 membered heteroalkynyl containing 1 to 3 heteroatoms selected from nitrogen, oxygen, and sulfur, or two adjacent R₂ together with their attached carbon atoms form a group selected from the group consisting of: substituted or unsubstituted C₃₋₁₀ cycloalkyl, substituted or unsubstituted C₆₋₁₀ aryl, substituted or unsubstituted 3-10 membered heterocyclyl containing 1 to 3 heteroatoms selected from nitrogen, oxygen, and sulfur, and substituted or unsubstituted 5-10 membered heteroaryl containing 1 to 6 heteroatoms selected from nitrogen, oxygen, and sulfur;
wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, each R₂ is independently selected from the group consisting of: H, substituted or unsubstituted C₁₋₆ alkyl, substituted or unsubstituted 1-6 membered heteroalkyl containing 1 to 3 heteroatoms selected from nitrogen, oxygen, and sulfur, substituted or unsubstituted C₂₋₆ alkenyl, substituted or unsubstituted 2-6 membered heteroalkenyl containing 1 to 3 heteroatoms selected from nitrogen, oxygen, and sulfur, substituted or unsubstituted C₂₋₆ alkynyl, substituted or unsubstituted 2-6 membered heteroalkynyl containing 1 to 3 heteroatoms selected from nitrogen, oxygen, and sulfur, or two adjacent R₂ together with their attached carbon atoms form a group selected from the group consisting of: substituted or unsubstituted C₃₋₇ cycloalkyl, substituted or unsubstituted C₆₋₁₀ aryl, substituted or unsubstituted 3-7 membered heterocyclyl containing 1 to 3 heteroatoms selected from nitrogen, oxygen, and sulfur, and substituted or unsubstituted 5-7 membered heteroaryl containing 1 to 3 heteroatoms selected from nitrogen, oxygen, and sulfur;
wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, each R₂ is independently selected from the group consisting of: H, substituted or unsubstituted C₁₋₆ alkyl, substituted or unsubstituted 1-6 membered heteroalkyl containing 1 to 3 heteroatoms selected from nitrogen, oxygen, and sulfur, or two adjacent R₂ together with their attached carbon atoms form a group selected from the group consisting of: substituted or unsubstituted C₃₋₇ cycloalkyl, substituted or unsubstituted phenyl;
wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, each Y and Z are independently selected from the group consisting of: null, substituted or unsubstituted C₁₋₁₀ alkylene, substituted or unsubstituted 1-10 membered heteroalkylene containing 1 to 3 heteroatoms selected from nitrogen, oxygen, and sulfur, substituted or unsubstituted C₃₋₁₀ cycloalkylene, substituted or unsubstituted C₅₋₁₀ arylene, substituted or unsubstituted Ar1-L-Ar2, and substituted or unsubstituted 5-10 membered heteroarylene;
L is selected from the group consisting of: null, O, S, NH, C₁₋₆ alkylene, and phenylene;
Ar1 and Ar2 are each independently null, phenylene, or 5-membered heteroarylene.

In another preferred embodiment, each Y and Z are independently null, substituted or unsubstituted C₁₋₇ alkylene, substituted or unsubstituted 1-7 membered heteroalkylene containing 1 to 3 heteroatoms selected from nitrogen, oxygen, and sulfur, substituted or unsubstituted C₃₋₇ cycloalkylene, substituted or unsubstituted phenylene, substituted or unsubstituted Ar1-L-Ar2, or substituted or unsubstituted 5-8 membered heteroarylene; L is selected from the group consisting of: null, O, S, NH, C₁₋₆ alkylene, and phenylene; Ar1 and Ar2 are each independently null, phenylene, or 5-membered heteroarylene; wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, X is selected from the group consisting of: Cl⁻, Br⁻, I⁻, SO₄²⁻, HSO₄⁻, PO₄³⁻, HOPO₃²⁻, (HO)₂PO₂⁻, R′CO₂⁻, ⁻O₂C(CH₂)ₙCO₂⁻ (n=0-20), ArCO₂⁻, R′SO₃⁻, ArSO₃⁻, R′PO₃⁻, ArPO₃⁻, alginate, citrate, aspartate, camphorate, camphorsulfonate, digluconate, glycerophosphate, fumarate, taurate, 2-hydroxyethanesulfonate, lactate, maleate, nicotinate, pamoate, pectate, thiocyanate, 3-phenylpropionate, picrate, and tartrate. Wherein, R′ is substituted or unsubstituted C₁₋₁₀ alkyl, and Ar is substituted or unsubstituted 5-7 membered aryl, or substituted or unsubstituted 5-7 membered heteroaryl containing 1-3 heteroatoms selected from N, O, and S. Preferably, X is chloride ion.

In another preferred embodiment, R′SO₃— is selected from CH₃SO₃⁻, PhSO₃⁻, and CH₃PhSO₃⁻.

In another preferred embodiment, R₁ is H, D, substituted or unsubstituted C₁₋₃ alkyl, and allyl; wherein, the “substituted” refers to the substitution of hydrogen atoms on the group by one or more C₁₋₄ alkyl or halogen.

In another preferred embodiment,

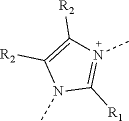

is selected from

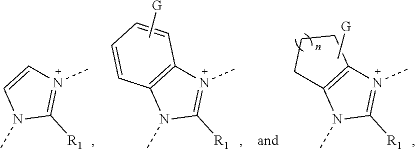

wherein, n=1, 2; G is H, or one or more groups selected from the group consisting of: C₁₋₆ alkyl, C₁₋₆ alkoxy, and C₁₋₆ alkoxycarbonyl.

In another preferred embodiment, each Y and Z are independently null, or a group that is substituted or unsubstituted and selected from the group consisting of:

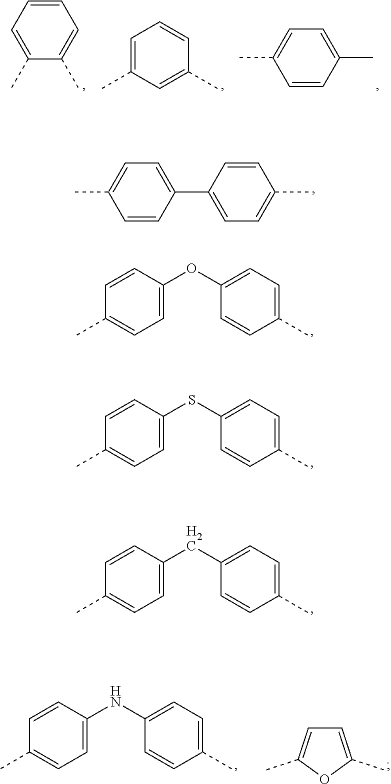

wherein, the “substituted” refers to the substitution of one or more hydrogen atoms on the group by a group selected from the group consisting of: C₁₋₆ alkyl, halogen, C₁₋₆ alkoxycarbonyl, and amido.

In another preferred embodiment, the halogen is fluorine, chlorine, bromine, or iodine.

In another preferred embodiment, each R₁ is independently selected from the group consisting of: H, methyl and ethyl.

In another preferred embodiment, each R₂ is independently hydrogen, or two adjacent R₂ together with their attached carbon atoms form a substituted or unsubstituted benzene ring, cyclopentyl, or cyclohexyl.

In another preferred embodiment, the compound is selected from the group consisting of:

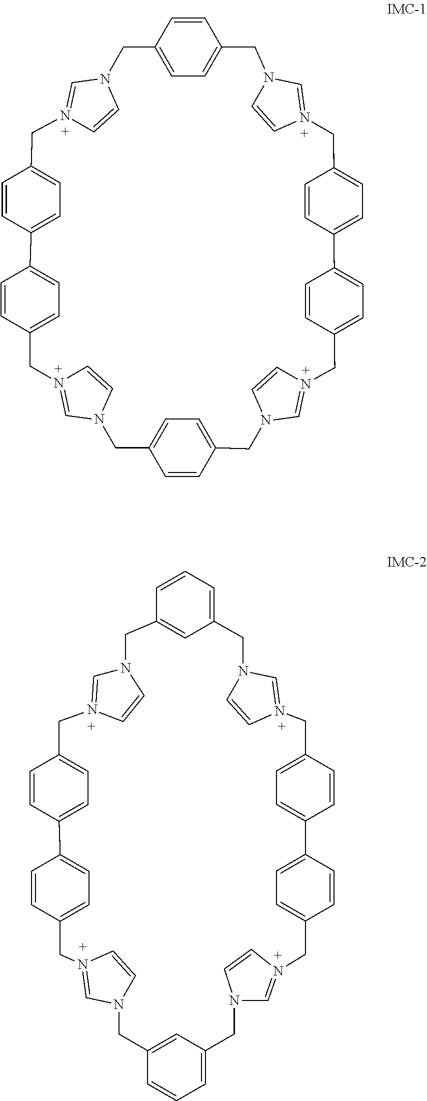

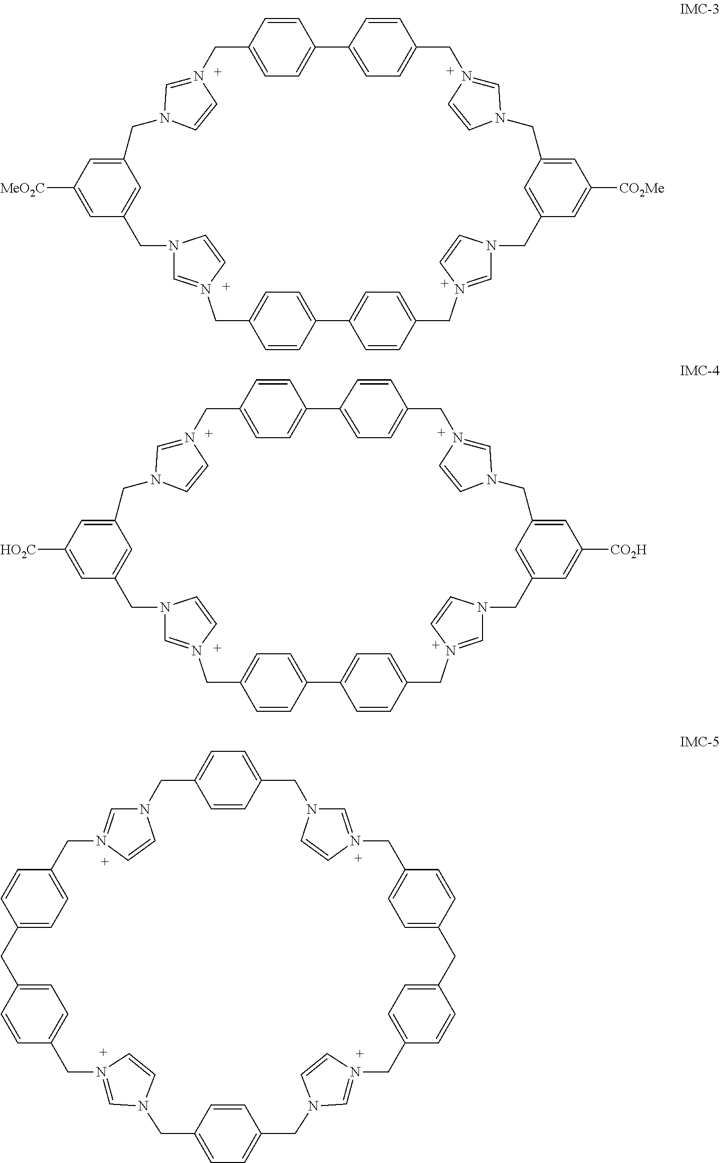

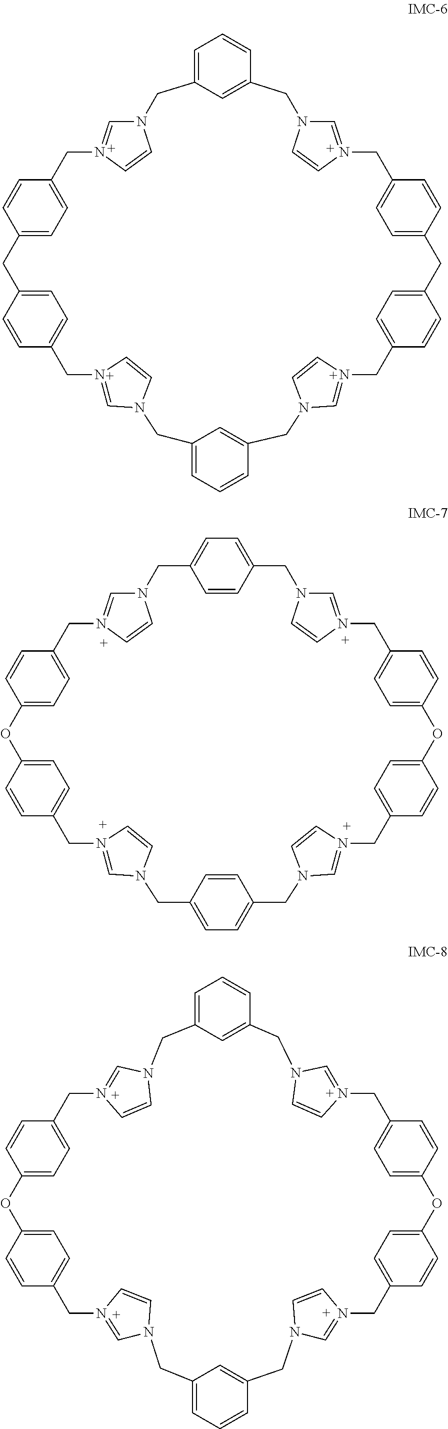

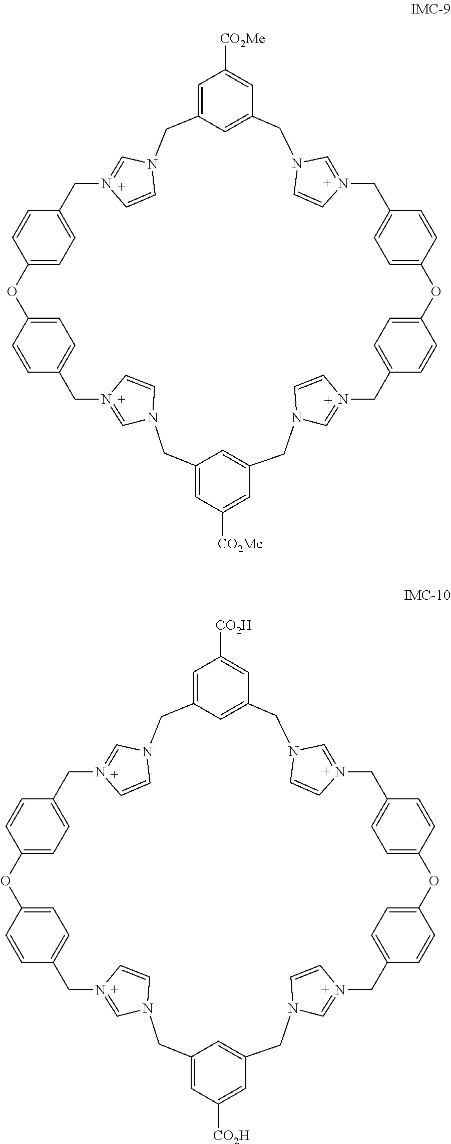

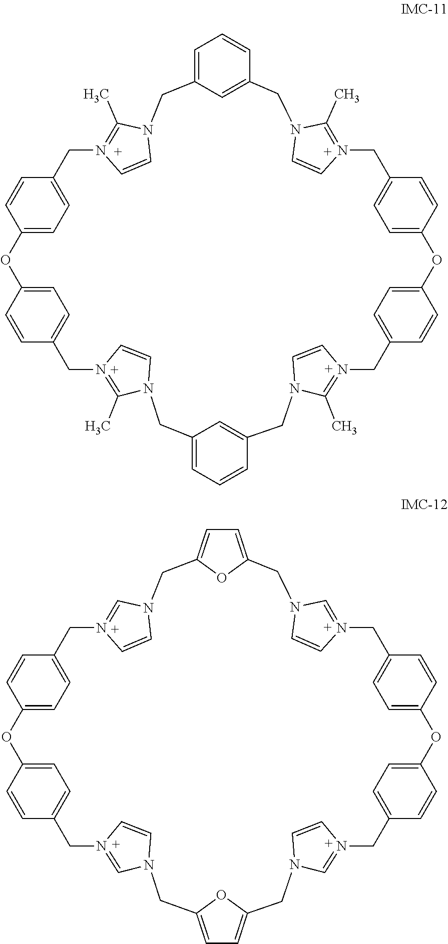

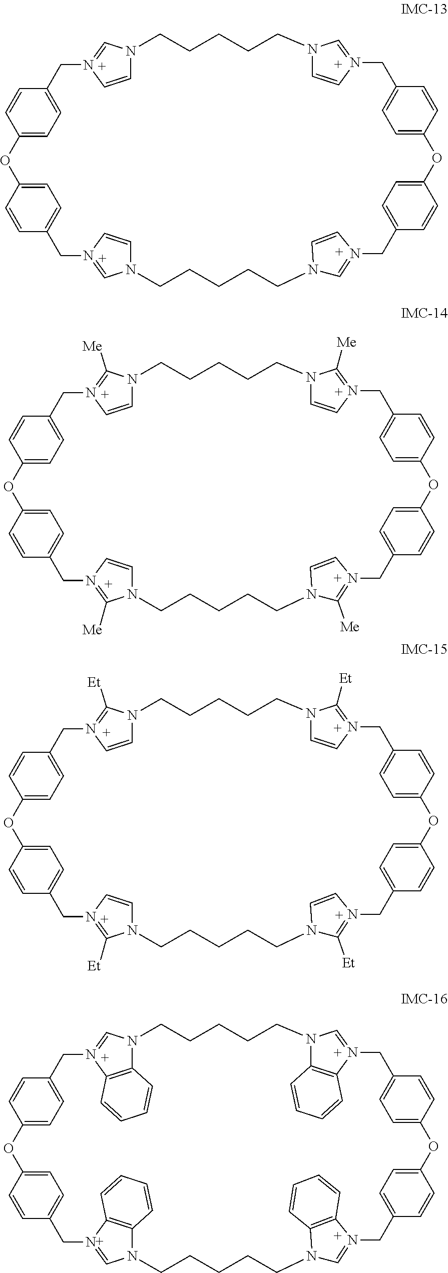

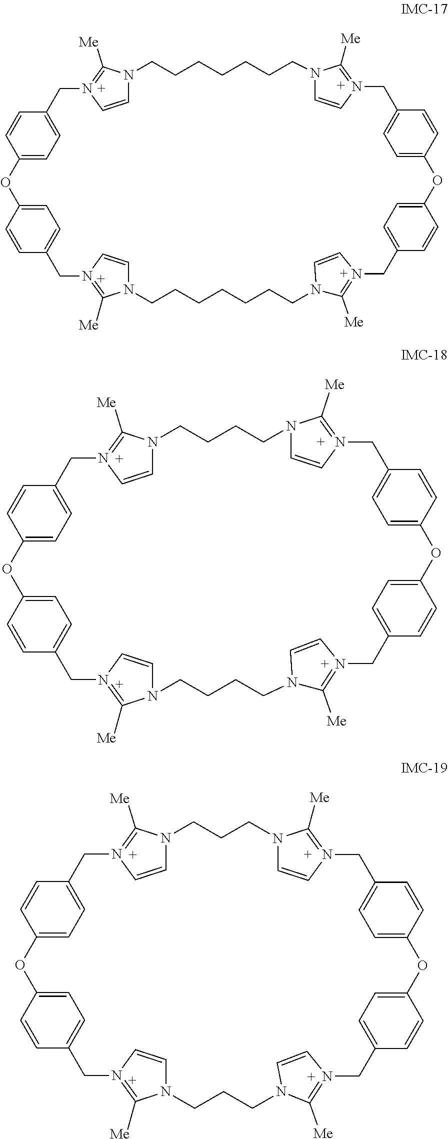

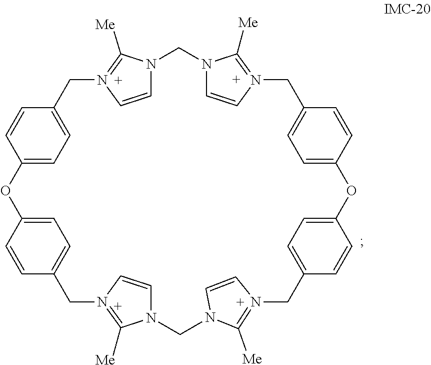

wherein, X is an anion paired with the tetravalent cationic imidazolium macrocycle; and the anion is selected from the group consisting of: Cl⁻, Br⁻, I⁻, SO₄²⁻, HSO₄⁻, PO₄³⁻, HOPO₃²⁻, (HO)₂PO₂⁻, R′CO₂⁻, ⁻O₂C(CH₂)ₙCO₂⁻ (n=0-20), ArCO₂⁻, R′SO₃⁻, ArSO₃⁻, R′PO₃⁻, ArPO₃⁻, alginate, citrate, aspartate, camphorate, camphorsulfonate, digluconate, glycerophosphate, fumarate, taurate, 2-hydroxyethanesulfonate, lactate, maleate, nicotinate, pamoate, pectate, thiocyanate, 3-phenylpropionate, picrate, and tartrate; wherein, R′ is substituted or unsubstituted C₁₋₁₀ alkyl, and Ar is substituted or unsubstituted C₅₋₇ aryl, or substituted or unsubstituted 5-7 membered heteroaryl containing 1-3 heteroatoms selected from N, O, and S.
wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, the compound is

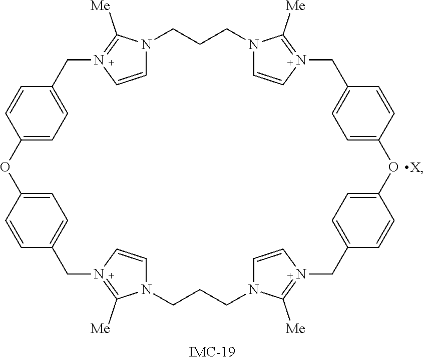

wherein, X is an anion paired with the tetravalent cationic imidazolium macrocycle; and the anion is selected from the group consisting of: Cl⁻, Br⁻, I⁻, SO₄²⁻, HSO₄⁻, PO₄³⁻, HOPO₃²⁻, (HO)₂PO₂⁻, R′CO₂⁻, ⁻O₂C(CH₂)~CO₂⁻ (n=0-20), ArCO₂⁻, R′SO₃⁻, ArSO₃⁻, R′PO₃⁻, ArPO₃⁻, alginate, citrate, aspartate, camphorate, camphorsulfonate, digluconate, glycerophosphate, fumarate, taurate, 2-hydroxyethanesulfonate, lactate, maleate, nicotinate, pamoate, pectate, thiocyanate, 3-phenylpropionate, picrate, and tartrate; wherein, R′ is substituted or unsubstituted C₁₋₁₀ alkyl, and Ar is substituted or unsubstituted C₅₋₇ aryl, or substituted or unsubstituted 5-7 membered heteroaryl containing 1-3 heteroatoms selected from N, O, and S.
wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

In another preferred embodiment, the compound is selected from the group consisting of:

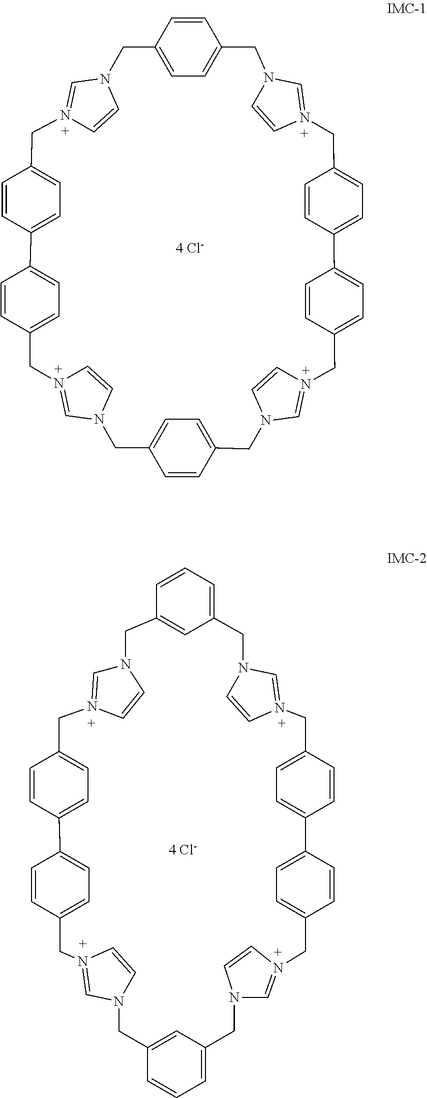

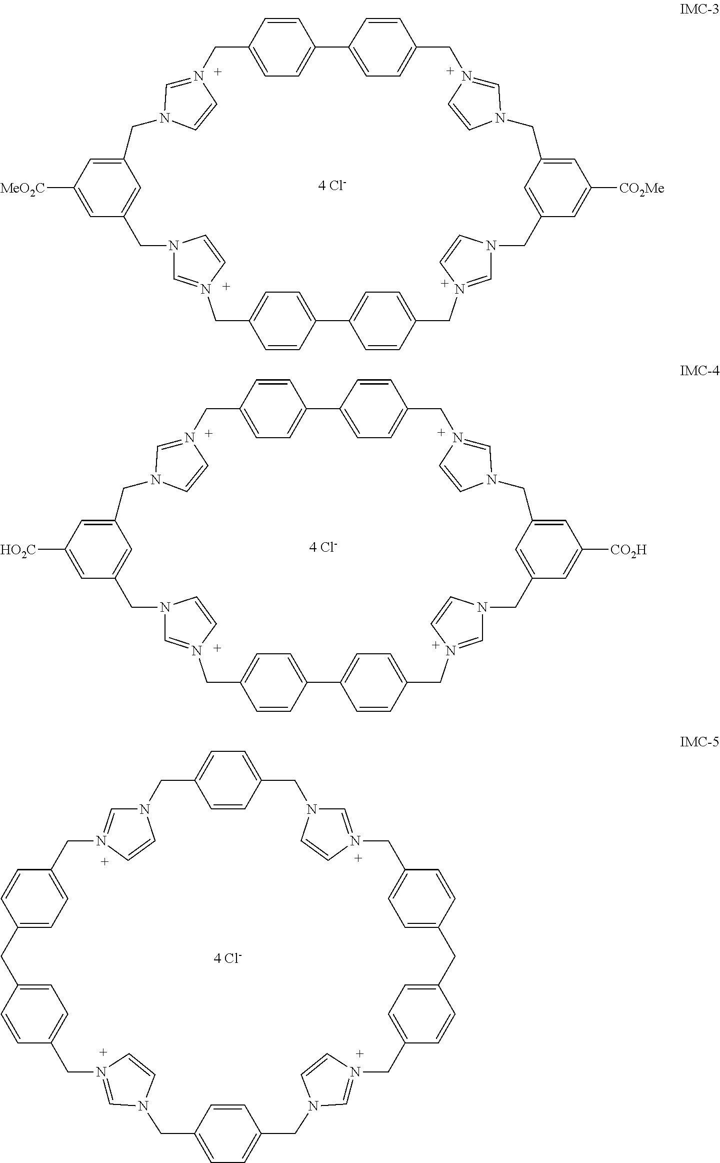

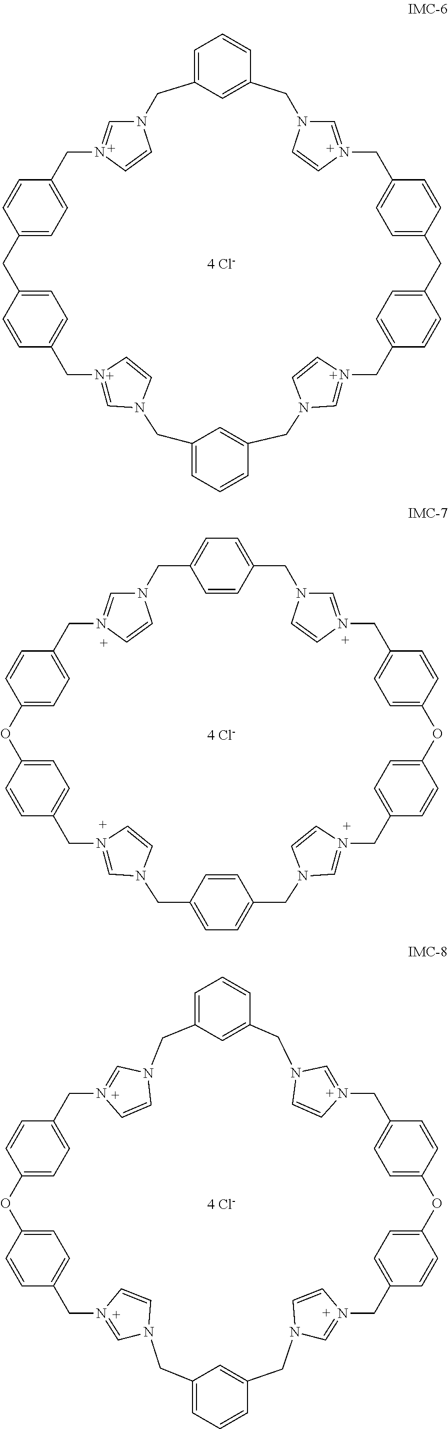

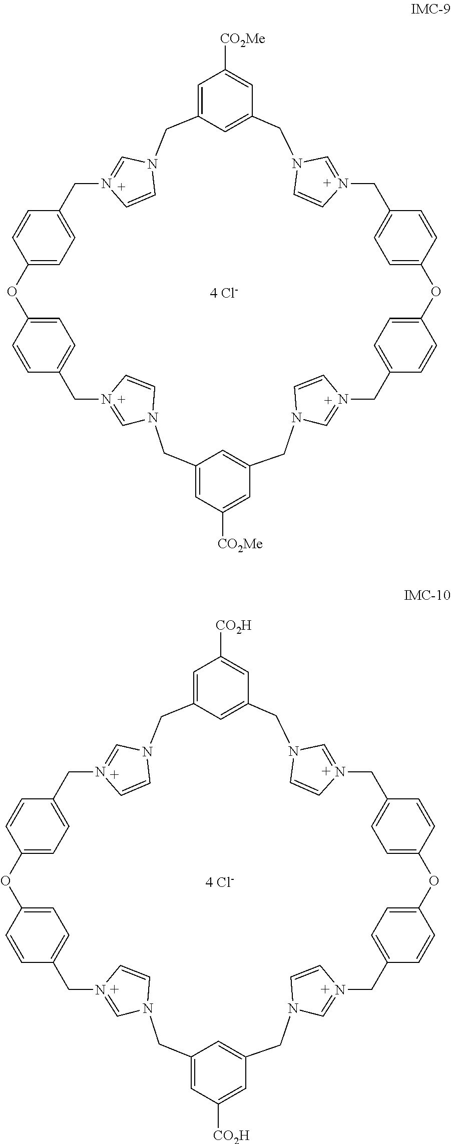

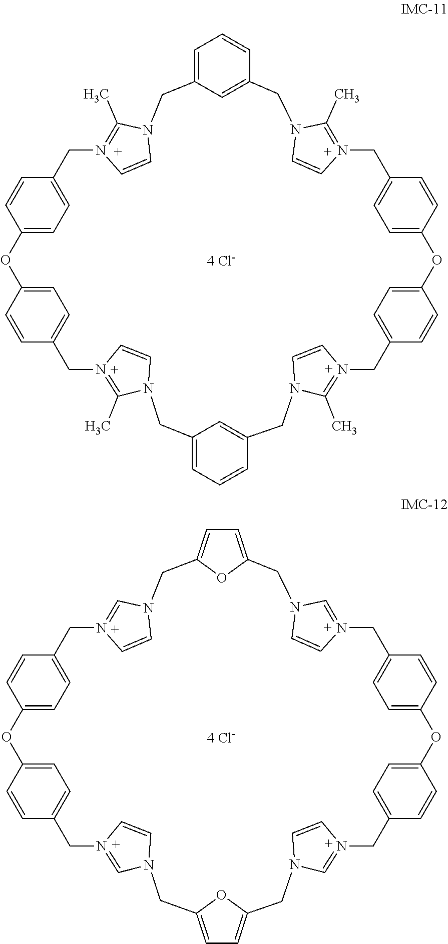

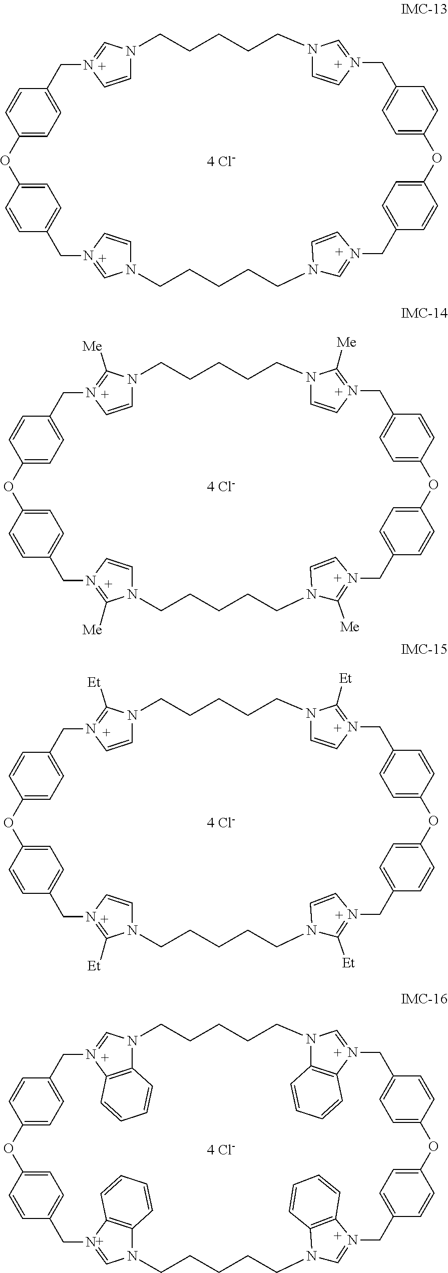

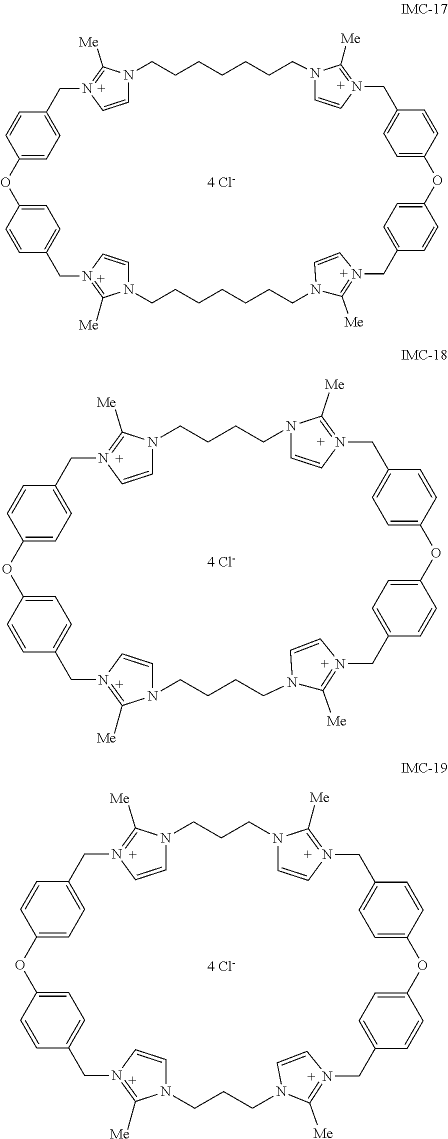

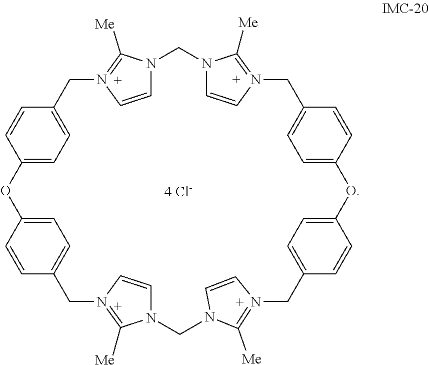

In another preferred embodiment, the compound is

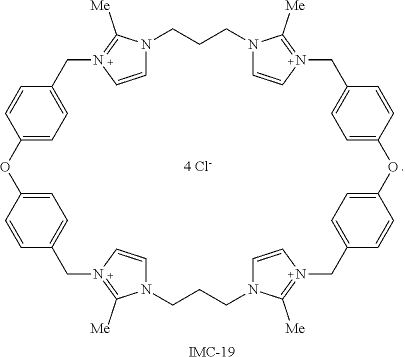

In the second aspect of the present invention, provided is a pharmaceutical composition, comprising:
(a) the compound according to the first aspect of the present invention, or the pharmaceutically acceptable salt, the hydrate or the solvate thereof as active ingredient; and (b) pharmaceutically acceptable carriers.

In another preferred embodiment, in the composition, component (a) accounts for 0.001-99.999 wt % of the total weight of the composition; preferably 0.01-99.99 wt %; more preferably 0.1-90 wt %.

In another preferred embodiment, the dosage form of the pharmaceutical composition is injection, tablets, capsules, pills, suspension, or emulsion.

In the third aspect of the present invention, provided is a pharmaceutical composition, comprising:
(a) the imidazolium salt macrocyclic compound according to the first aspect of the present invention as a first active ingredient;
(b) an ingredient having muscle relaxation effects as a second active ingredient that is selected from the group consisting of: rocuronium bromide, vecuronium bromide, cisatracurium besylate, pancuronium bromide, or a combination thereof; and
(c) pharmaceutically acceptable carriers or excipients.

In another preferred embodiment, the dosage form of the pharmaceutical composition is injection, tablets, capsules, pills, suspension, or emulsion.

In the fourth aspect of the present invention, provided is a method for preparing the compound according to the first aspect of the present invention, comprising steps of:

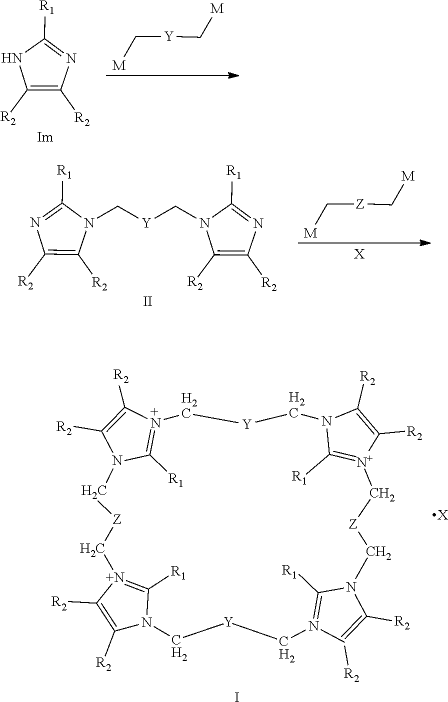

(1) Subjecting excess amount of imidazole unit Im and dihalogenated compound MCH₂YCH₂M to a substitution reaction to obtain imidazole intermediate;
(2) Subjecting the imidazole intermediate and dihalogenated compound MCH₂ZCH₂M to an intermolecular and intramolecular substitution reaction for ring closure to obtain a tetravalent cationic imidazolium salt macrocyclic compound;
(3) Subjecting the tetravalent cationic imidazolium salt macrocyclic compound to anion X exchange to obtain the corresponding salt;
wherein, each R₁, R₂, X, Y, and Z are as described in the first aspect of the present invention; M is halogen.

In another preferred embodiment, the dihalogenated compound MCH₂YCH₂M and the dihalogenated compound MCH₂ZCH₂M can interchange with each other to react.

In another preferred embodiment, the dihalogenated compound MCH₂YCH₂M and the dihalogenated compound MCH₂ZCH₂M are the same or different.

In the fifth aspect of the present invention, provided is a use of the compound according to the first aspect of the present invention, the pharmaceutical composition according to the second aspect of the present invention, or the pharmaceutical composition according to the third aspect of the present invention as muscle relaxant.

In the sixth aspect of the present invention, provided is a kit, comprising:
(a) a first container, and the compound according to the first aspect of the present invention, or the pharmaceutically acceptable salt, the hydrate, or the solvate thereof as the active ingredient that is placed in the first container, and/or
(b) a second container, and a muscle relaxant antagonist placed in the second container: Neostigmine, Sugammadex Sodium, or a combination thereof;
(c) a n-th container and the n-th pharmaceutical ingredient placed in the n-th container, wherein n is any positive integer from 3 to 30; wherein, the n-th pharmaceutical ingredient is a compound according to the first aspect of the present invention, or a pharmaceutical composition according to the second aspect of the present invention, or a pharmaceutical composition according to the third aspect of the present invention; and/or
(d) optional instructions.

In another preferred embodiment, the kit is used for relaxing skeletal muscles.

In the seventh aspect of the present invention, provided is a method for relaxing skeletal muscles, comprising steps of: administering to a subject in need thereof a therapeutically effective amount of the compound according to the first aspect of the present invention, the pharmaceutical composition according to the second aspect of the present invention, or the pharmaceutical composition according to the third aspect of the present invention, thereby relaxing the skeletal muscles.

In another preferred embodiment, the subject is mammal, preferably rodent or primate mammal, and more preferably mouse, rat, or human.

In another preferred embodiment, the method is in vitro.

In another preferred embodiment, the method is non-diagnostic and non-therapeutic.

It should be understood that, within the scope of the present invention, each of the above technical features of the present invention and each of the technical features specifically described in the following (such as the embodiments) can be combined with each other to constitute a new or preferred technical solution. Due to space limitations, it will not be repeated herein.

### EMBODIMENTS FOR CARRYING OUT THE INVENTION

After extensive and in-depth research, the inventor has provided for the first time a structurally novel macrocyclic compound incorporating four imidazolium salt groups, and used it as a muscle relaxant. The compound of the present invention carries positive charges and has rapid and long-acting muscle relaxation function. The SD rat animal model experiment showed that the imidazolium salt macrocyclic compound of the present invention has high muscle relaxation activity, and the increase in dose can prolong the muscle relaxation time to 5 hours. In theory, when converting to an adult dose, it can achieve muscle relaxation for over 10 hours, covering over 95% of surgical patients and achieving rapid onset. Based on this, the inventors completed the present invention.

### Terms

Unless otherwise defined, all technical and scientific terms used herein have the same meaning as would normally be understood by those of ordinary skill in the art to which the invention belongs.

As used herein, when used in reference to a specific enumerated value, term “about” means that the value may change by no more than 1% from the enumerated value. For example, as used herein, the expression “about 100” includes all values between 99 and 101 and (e.g., 99.1, 99.2, 99.3, 99.4, etc.).

As used herein, term “contain” or “include (comprise)” may be open, semi-closed, and closed. In other words, said term also includes “consisting essentially of” or “consisting of”.

As used herein, “C₁₋₂₀ alkyl” represents a linear or branched chain alkyl —(CH₂)ₙ₋₁CH₃ (n=1-20) having 1 to 20 carbon atoms, such as methyl, ethyl, propyl, isopropyl, N-butyl, isobutyl, tert-butyl and the like.

As used herein, “1-20 membered heteroalkyl” refers to a group where one or more carbon atom in the chain of C₁₋₂₀ alkyl is replaced by heteroatoms selected from nitrogen, oxygen, and sulfur. For example, 1-8 membered heteroalkyl refer to CH₃—CH₂—CH₂—CH₂—O—CH₂—CH₂—CH₂—, or CH₃—CH₂—CH₂—CH₂—O—CH₂—CH₂—O— and the like.

As used herein, “2-20-membered heteroalkenyl” refers to a group where one or more carbon atoms in the chain of C₂₋₂₀ alkenyl are replaced by heteroatoms selected from nitrogen, oxygen, and sulfur. For example, 2-7-membered heteroalkenyl refers to groups such as CH₃—CH₂—CH₂—O—CH₂—CH═CH—, or CH₃—S—CH₂—CH₂O—CH═CH—, and the like.

As used herein, “2-20-membered heteroalkynyl” refers to a group where one or more carbon atoms in the chain of C₂₋₂₀ alkynyl are replaced by heteroatoms selected from nitrogen, oxygen, and sulfur. For example, 2-7-membered heteroalkynyl refers to groups such as CH₃—CH₂—CH₂—O—CH₂—C≡C—, or CH₃—S—CH₂—CH₂—O—C≡C—, and the like.

As used herein, “C₃₋₂₀ cycloalkyl” refers to a cyclic alkyl group or a cyclic alkyl group with side chains containing 3-20 carbon atoms, such as cyclopropyl, cyclobutyl, cyclopentyl, cyclohexyl, etc.

As used herein, the term “active ingredient” refers to an imidazolium salt macrocyclic compound, or a pharmaceutically acceptable salt, a hydrate, or a solvate thereof. It should be understood that active ingredients also include mixtures of such compounds.

As used herein, the term “hydrate” refers to a complex formed by the coordination of the compound of the present invention with water.

As used herein, the term “solvate” refers to a complex formed by the coordination of the compound of the present invention with solvent molecules in any specific ratio.

As used herein, the term “aryl” refers to monovalent aromatic carbocyclyl having from 6 to 20 (preferably from 6 to 14) carbon atoms, which has a monocyclic (such as phenyl) or fused (such as naphthyl or anthryl) ring. If the connecting point is located on the aromatic atom, the fused ring may be non-aromatic (such as 2-benzoxazolone, 2H-1,4-benzoxazin-3(4H)-one-7-yl, etc.). Preferably, aryl includes phenyl and naphthyl.

As used herein, the term “cycloalkyl” refers to a cyclic alkyl having 3-12 (preferably 3 to 10) carbon atoms and monocyclic and polycyclic ring (including fused, bridged and spiro ring system). In a fused ring system, one or more rings can be cycloalkyl, heterocyclyl, aryl, or heteroaryl, as long as the connecting site is through a cyclic alkyl ring. Examples of suitable cycloalkyl include: for example, adamantyl, cyclopropyl, cyclobutyl, cyclopentyl, and cyclooctyl.

As used herein, the term “heteroaryl” refers to an aromatic group having 2 to 10 carbon atoms and 1 to 4 heteroatoms selected from oxygen, nitrogen, and sulfur in the ring structure. Such heteroaryl can be monocyclic (such as pyridyl or furyl) or fused (such as indolizinyl or benzothienyl), wherein, the fused ring can be non-aromatic and/or contain one heteroatom, as long as the connecting point is through an aromatic heteroaryl atom. In one embodiment, the ring atom nitrogen and/or sulfur of the heteroaryl is optionally oxidized to N-oxide (N—O), sulfonyl or sulfonyl. Preferable heteroaryl includes pyridinyl, pyrrolyl, indolyl, thienyl, and furyl. In one embodiment, the heteroaryl refers to 3-14 membered heteroaryl, preferably 5-12 membered heteroaryl.

As used herein, the term “substituted heteroaryl” refers to a heteroaryl substituted with from 1 to 5, preferably from 1 to 3, and more preferably from 1 to 2 substituents, wherein the substituent is the same as those defined in the substituted aryl.

The animal activity test and acute toxicity test in the present invention were carried out on SD rats.

As used herein, unless otherwise specified, the “substituted” refers to the substitution of one or more hydrogens on a group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

As used herein, the compound of formula I of the present invention consists of a tetravalent cationic imidazolium salt macrocycle and anion X that charge-balances the tetravalent cationic imidazolium salt macrocycle; wherein, X is one or more anions selected from the group consisting of: Cl⁻, Br⁻, I⁻, SO₄²⁻, HSO₄⁻, PO₄³⁻, HOPO₃²⁻, (HO)₂PO₂⁻, R′CO₂⁻, ⁻O₂C(CH₂)ₙCO₂⁻ (n=0-20), ArCO₂⁻, R′SO₃⁻, ArSO₃⁻, R′PO₃⁻, ArPO₃⁻, alginate, citrate, aspartate, camphorate, camphorsulfonate, digluconate, glycerophosphate, fumarate, taurate, 2-hydroxyethanesulfonate, lactate, maleate, nicotinate, pamoate, pectate, thiocyanate, 3-phenylpropionate, picrate, and tartrate; wherein, R′ is substituted or unsubstituted C₁₋₁₀ alkyl, and Ar is substituted or unsubstituted C₅₋₇ aryl, or substituted or unsubstituted 5-7 membered heteroaryl containing 1-3 heteroatoms selected from N, O, and S; wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl. Sum of the valence states of anions represented by X is equal to 4, thus being charge-balanced with the tetravalent imidazolium salt macrocycle. The anions represented by X can be 4 monovalent anions, 2 divalent anions, or 1 divalent anion and 2 monovalent anions; and can also be a monovalent anion and a trivalent anion; wherein, each monovalent anions in the 4 monovalent anions can be the same or different, and each divalent anions in the 2 divalent anions can be the same or different.

### Neuromuscular Blocking Agents or Muscle Relaxants

Neuromuscular blocking agents or skeletal muscle relaxants (also known as muscle relaxants) are widely used in clinical practice to relax skeletal muscles, thereby benefiting intubation, or promoting surgical conditions. Muscle relaxants are categorized into depolarizing and non-depolarizing types based on their different mechanisms of action: depolarizing muscle relaxants bind to N2 cholinocepters on the motor nerve end plate membrane, weakening or disappearing the response of muscle cells to acetylcholine; non-depolarizing muscle relaxants compete with acetylcholine for N2 cholinocepters on the motor end plate membrane of skeletal muscle, leading to muscle relaxation. The non-depolarizing muscle relaxants commonly used in clinical practice are classified into amino steroid derivatives (such as rocuronium bromide, vecuronium bromide, and pancuronium bromide) and benzylisoquinoline derivatives (such as cisatracurium besylate) based on their chemical structures.

Unlike the tetraalkyl cationic ammonium salt derivatives used clinically as muscle relaxants, the present invention provides a macrocyclic compound represented by Formula I incorporating four imidazolium salt groups, or a pharmaceutically acceptable salt, a hydrate, or a solvate thereof.

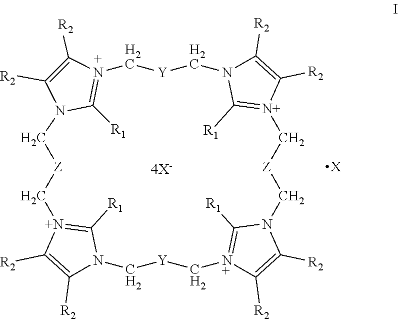

wherein, each R₁, R₂, Z, Y, and X are as described in the first aspect of the present invention.

In another preferred embodiment, R₁ is H, D, substituted or unsubstituted C₁₋₃ alkyl, and allyl; wherein, the “substituted” refers to the substitution of hydrogen atoms on the group by one or more C₁₋₄ alkyl or halogen.

In another preferred embodiment,

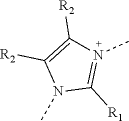

is selected from

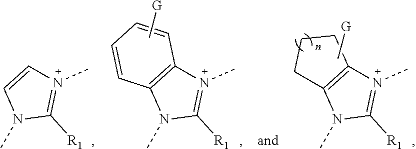

wherein, n=1, 2; G is H, or one or more groups selected from the group consisting of: C₁₋₆ alkyl, C₁₋₆ alkoxy, and C₁₋₆ alkoxycarbonyl.

In another preferred embodiment, each Y and Z are independently null, or a substituted or unsubstituted group selected from the group consisting of:

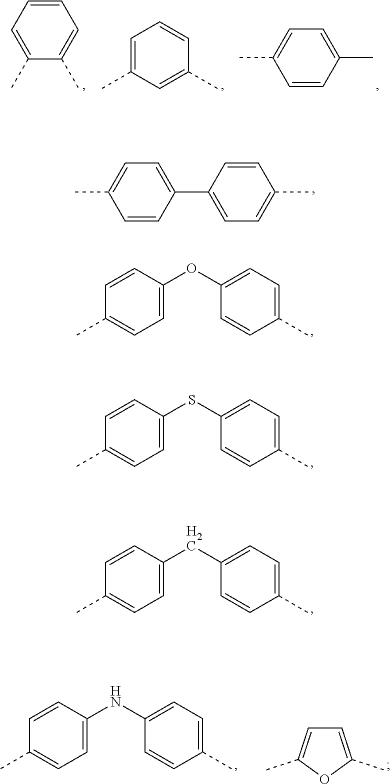

wherein, the “substituted” refers to the substitution of one or more hydrogen atoms on the group by a group selected from the group consisting of: C₁₋₆ alkyl, halogen, C₁₋₆ alkoxycarbonyl, and amido.

In another preferred embodiment, the halogen is fluorine, chlorine, bromine, or iodine.

In another preferred embodiment, each R₁ is independently selected from the group consisting of: H, methyl and ethyl.

In another preferred embodiment, each R₂ is independently hydrogen, or two adjacent R₂ together with their attached carbon atoms form a substituted or unsubstituted benzene ring, cyclopentyl, or cyclohexyl.

### Preparation Method

The preparation method of compound of formula (I) of the present invention will be further described in the following and these specific methods do not constitute any limitation on the present invention. The compound of the present invention can also be conveniently prepared by combining various synthesis methods described in this specification or known in the art, and such combinations can be easily carried out by those skilled in the art to which the present invention belongs.

The present invention discloses a macrocyclic compound incorporating four imidazolium salts, and the preparation method therefor. Animal experiments support that such compounds have efficient neuromuscular blocking activity and exhibit rapid onset and long-acting characteristics.

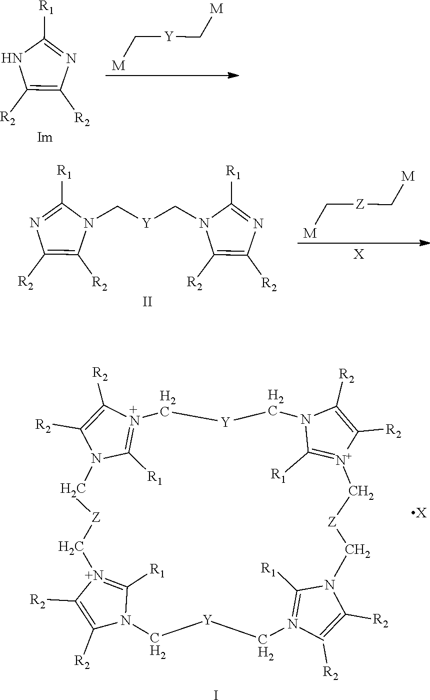

(1) Excess amount of imidazole unit Im and dihalogenated compound MCH₂YCH₂M undergo substitution reaction to obtain imidazole intermediate;
(2) The imidazole intermediate and dihalogenated compound MCH₂ZCH₂M undergo intermolecular and intramolecular substitution reaction for closing ring to obtain tetravalent cationic imidazolium salt macrocyclic compound;
(3) The tetravalent cationic imidazolium salt macrocyclic compound undergoes anion X exchange to obtain the corresponding salt;
wherein, each R₁, R₂, X, Y, and Z are as described in the first aspect of the present invention; M is halogen.

In another preferred embodiment, the dihalogenated compound MCH₂YCH₂M and the dihalogenated compound MCH₂ZCH₂M can react interchangeably.

In another preferred embodiment, the dihalogenated compound MCH₂YCH₂M is the same as or different from the dihalogenated compound MCH₂ZCH₂M.

### Pharmaceutical Composition

The pharmaceutical composition of the present invention comprises a safe and effective amount of the compound of the present invention, or a pharmaceutically acceptable salt thereof and pharmaceutically acceptable excipients or carriers. Wherein “safe and effective amount” refers to an amount of compound which is sufficient to significantly improve the condition, and not to generate severe side effects. Generally, the pharmaceutical composition contains 1-2000 mg of the compound of the present invention per dose, preferably, 10-1000 mg the compound of the present invention per dose. Preferably, the “one dose” is one capsule or one pill or one injection.

As used herein, the “active ingredient” refers to the compound of formula I of the present invention, or a pharmaceutically acceptable salt, a hydrate or a solvate thereof.

As used herein, the term “pharmaceutically acceptable salt” refers to non-toxic acid or alkaline earth metal salts of the compound of formula I. These salts can be prepared in situ during the final separation and purification of the compound of formula I, or by reacting suitable organic or inorganic acids or bases with basic or acidic functional groups, respectively. Representative salts include, but are not limited to: acetate, adipate, alginate, citrate, aspartate, benzoate, benzenesulfonate, bisulfate, butyrate, camphorate, camphorsulfonate, digluconate, cyclopentane propionate, dodecyl sulfate, ethanesulfonate, glucose enanthate, glycerophosphate, hemisulphate, enanthate, caproate, fumarate, hydrochloride, hydrobromide, hydroiodate, 2-hydroxyethanesulfonate, lactate, maleate, methanesulfonate, nicotinate, 2-naphthylsulfonate, oxalate, pamoate, pectate, thiocyanate, 3-phenylpropionate, picrate, pivalate, propionate, succinate, sulfate, tartrate, thiocyanate, p-toluenesulfonate, and undecanoate. In addition, nitrogen-containing basic group can be quaternized by the following reagents: alkyl halides, such as chlorides, bromides, and iodides of methyl, ethyl, propyl, and butyl; dialkyl sulfates, such as dimethyl, diethyl, dibutyl, and dipentyl sulfates; long chain halides such as chlorides, bromides, and iodides of decyl, lauryl, myristyl, and stearyl; arylalkyl halides such as benzyl and phenylethyl bromides. Thus, water-soluble, oil soluble, or dispersible products are obtained. Examples of acids that can be used to form pharmaceutically acceptable acid addition salts include inorganic acids such as hydrochloric acid, sulfuric acid, and phosphoric acid, as well as organic acids such as oxalic acid, maleic acid, methanesulfonic acid, succinic acid, and citric acid. Alkali addition salts can be prepared in situ during the final separation and purification of the compound of formula I, or by reacting the carboxylic acid moiety with suitable bases (such as pharmaceutically acceptable hydroxides, carbonates, or bicarbonates of metal cation) or ammonia, or organic primary, secondary, or tertiary amines. Pharmaceutically acceptable salts include, but are not limited to, cations based on alkali metals and alkaline earth metals such as sodium, lithium, potassium, calcium, magnesium, aluminum salts, as well as non-toxic ammonium, quaternary ammonium, and amine cations, including but not limited to: ammonium, tetramethylammonium, tetraethylammonium, methylamine, dimethylamine, trimethylamine, triethylamine, ethylamine, etc. Other representative organic amines used to form base addition salts include diethylamine, ethylenediamine, ethanolamine, diethanolamine, piperazine, etc.

“Pharmaceutically acceptable carrier” means one or more compatible solid or liquid fillers, or gelatinous materials which are suitable for human use and should be of sufficient purity and sufficiently low toxicity. “Compatible” herein refers to the ability of each component of a composition can be mixed with the compound of the present invention and can be mixed with each other without appreciably reducing the efficacy of the compound. Examples of pharmaceutically acceptable carrier include cellulose and derivatives thereof (such as sodium carboxymethylcellulose, sodium ethylcellulose, cellulose acetate, etc.), gelatin, talc, solid lubricant (such as stearic acid, magnesium stearate), calcium sulfate, vegetable oil (such as soybean oil, sesame oil, peanut oil, olive oil, etc.), polyol (such as propylene glycol, glycerol, mannitol, sorbitol, etc.), emulsifier (such as Tween®), wetting agent (such as lauryl sodium sulfate), colorant, flavoring, stabilizer, antioxidant, preservative, pyrogen-free water, etc.

Suitable pharmaceutically acceptable carriers or excipients include: treatment agents and drug delivery modifiers and promoters, such as calcium phosphate, magnesium stearate, talc, monosaccharides, disaccharides, starch, gelatin, cellulose, sodium methylcellulose, carboxymethyl cellulose, glucose, hydroxypropyl-B-cyclodextrins, polyvinylpyrrolidone, low melting point wax, ion exchange resins, and any combination of two or more thereof. Liquid and semi-solid excipients can be selected from glycerol, propylene glycol, water, ethanol, and various oils, including petroleum, animal oil, vegetable oil, or synthetic sources, such as peanut oil, soybean oil, mineral oil, sesame oil, etc. Preferred liquid carriers, especially for injectable solutions, include water, saline, glucose aqueous solutions, and ethylene glycol. Other suitable pharmaceutically acceptable excipients are described in Remington's Pharmaceutical Sciences, Mack Pub. Co, New Jersey (1991), which is incorporated in this article by reference.

The pharmaceutical composition is injections, capsules, tablets, pills, powders or granules.

There is no special limitation of administration mode for the compound or pharmaceutical compositions of the present invention, and the representative administration mode includes (but is not limited to): oral, intratumoral, rectal, parenteral (intravenous, intramuscular or subcutaneous), and topical administration.

The solid dosage forms used for oral administration include capsules, tablets, pills, powders, and granules. In these solid dosage forms, the active compounds are mixed with at least one conventional inert excipient (or carrier), such as sodium citrate or dicalcium phosphate, or mixed with any of the following components: (a) fillers or compatibilizer, for example, starch, lactose, sucrose, glucose, mannitol and silicic acid; (b) binders, for example, hydroxymethyl cellulose, alginates, gelatin, polyvinylpyrrolidone, sucrose and arabic gum; (c) humectant, such as, glycerol; (d) disintegrating agents such as agar, calcium carbonate, potato starch or tapioca starch, alginic acid, certain composite silicates, and sodium carbonate; (e) dissolution-retarding agents, such as paraffin; (f) absorption accelerators, for example, quaternary ammonium compounds; (g) wetting agents, such as cetyl alcohol and glyceryl monostearate; (h) adsorbents, for example, kaolin; and (i) lubricants such as talc, stearin calcium, magnesium stearate, solid polyethylene glycol, sodium lauryl sulfate, or the mixtures thereof. In capsules, tablets, and pills, the dosage form may also include buffers.

Solid dosage forms such as tablets, sugar pills, capsules, pills, and granules can be prepared using coating and shell materials, such as casings and other materials well-known in the art. They may contain opacifiers, and the release of active compounds or compounds in the composition can be delayed in a certain part of the digestive tract. Examples of embedding components that may be employed are polymeric substances and waxes. If necessary, the active compound may also be formed into a microcapsules with one or more of the above excipients.

Liquid dosage forms for oral administration include pharmaceutically acceptable lotion, solutions, suspensions, syrups or tinctures. In addition to the active compounds, the liquid dosage forms may contain any conventional inert diluents known in the art such as water or other solvents, solubilizers and emulsifiers, for example, ethanol, isopropanol, ethyl carbonate, ethyl acetate, propylene glycol, 1,3-butanediol, dimethyl formamide, as well as oil, in particular, cottonseed oil, peanut oil, corn germ oil, olive oil, castor oil and sesame oil, or the combination thereof.

In addition to these inert diluents, the composition can also include additives such as wetting agents, emulsifiers and suspensions, sweeteners, flavoring agents, and spices.

In addition to active compounds, suspensions can include suspending agents such as ethoxylated isooctadecanol, polyoxyethylene sorbitol and dehydrated sorbitol esters, microcrystalline cellulose, methanol aluminum and agar, or mixtures of these substances.

Compositions for parenteral injection may include physiologically acceptable sterile aqueous or anhydrous solutions, dispersion liquid, suspensions or lotions, and sterile powders for re dissolution into sterile injectable solutions or dispersions. Suitable aqueous and non-aqueous carriers, diluents, solvents or excipients include water, ethanol, polyols, and their suitable mixtures.

The dosage forms of the compounds of the present invention used for local administration include ointments, powders, patches, sprays, and inhalants. The active ingredients are mixed under sterile conditions with physiologically acceptable carriers and any preservatives, buffers, and propellants if necessary.

The compound of the present invention may be administered alone or in combination with other pharmaceutically acceptable compound (such as antitumor drugs).

The treatment method of the present invention can be administered alone or in combination with other treatment methods or drugs.

When the pharmaceutical compositions are used, a safe and effective amount of compound of the present invention is applied to a mammal (such as human) in need of, wherein the dose of administration is a pharmaceutically effective dose. For a person weighed 60 kg, the daily dose is usually 1-2000 mg, preferably 50-1000 mg. Of course, the particular dose should also depend on various factors, such as the route of administration, patient healthy status, which are within the skills of an experienced physician.

Compared with the existing technologies, the main advantages of the present invention include:
(1) The imidazolium salt macrocyclic compound of the present invention has high muscle relaxant activity and high safety.
(2) The imidazolium salt macrocyclic compound of the present invention can provide rapid and long-acting non-depolarizing muscle relaxation, which meets the clinical needs.

The present invention was further described hereafter in combination with specific embodiments. It should be understood that these examples are only used to illustrate the invention and not to limit the scope of the invention. The experimental methods with no specific conditions described in the following examples are generally performed under the conventional conditions, for example, according to J. Sambrook et al., Molecular Cloning: A Laboratory Manual, 3rd Edition, Science Press, 1989, or according to the manufacture's instructions. Unless otherwise stated, percentages and parts are by weight.

Unless otherwise defined, all professional and scientific terms used in the text have the same meanings as those familiar to those skilled in the art. In addition, any methods and materials similar or equal to the recorded content can be applied to the methods of the present invention. The preferred embodiments and materials described herein are for exemplary purposes only.

### Example 1

Taking IMC-1 as an example, the imidazolium salt macrocyclic compound of the present invention uses the following general preparation route:

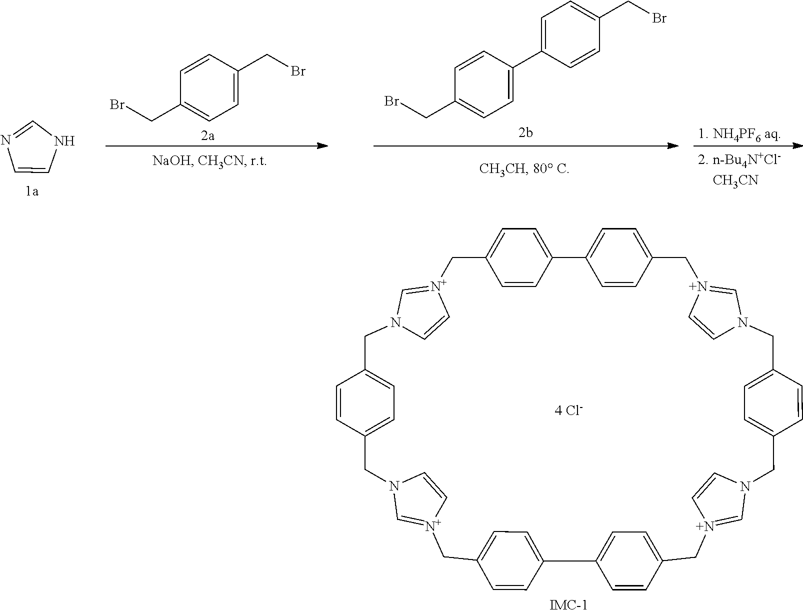 [SMILES: c1c[nH]cn1 ; BrCc1ccc(CBr)cc1 ; BrCc1ccc(-c2ccc(CBr)cc2)cc1 ; c1cc2ccc1Cn1cc[n+](c1)Cc1ccc(cc1)-c1ccc(cc1)C[n+]1ccn(c1)Cc1ccc(cc1)Cn1cc[n+](c1)Cc1ccc(cc1)-c1ccc(cc1)C[n+]1ccn(c1)C2]

Compound 1a (1.43 g, 21 mmol) was dissolved in 200 mL of acetonitrile, and the mixture was stirred at room temperature. Sodium hydroxide (1.6 g, 40 mmol) was added and the mixture continued stirring for 2 hours. Compound 2a (2.64 g, 10 mmol) was dissolved in 30 mL of acetonitrile, the above-mentioned acetonitrile solution was added dropwise and the mixture was stirred at room temperature for 2 hours. The mixture was filtered. The filtrate was spin-dried, dissolved thoroughly with 100 mL of dichloromethane, and washed repeatedly with deionized water until the washing solution was neutral. The dichloromethane solution was added with an appropriate amount of anhydrous sodium sulfate. After complete drying, the mixture was filtered. The filtrate was spin-dried to obtain 1.96 g of white solid (yield 82%) for the next reaction. The above-prepared intermediate (0.48 g, 2 mmol) was dissolved in 300 mL of acetonitrile, the solution was heated up to 80° C. and stirred thoroughly. Compound 2b (0.68 g, 2 mmol) was dissolved in 80 mL of acetonitrile, and added dropwise into the solution of intermediate in acetonitrile and the mixture was reacted at 80° C. for 48 hours. The reaction solution was evaporated under reduced pressure to remove the solvent, and the resulting crude product was separated and purified by column chromatography using methanol/water/saturated ammonium chloride aqueous solution (12:7:1) as the eluent. The eluent was collected, evaporated under reduced pressure to remove the solvent, and added with an appropriate amount of deionized water to prepare a saturated aqueous solution. Saturated ammonium hexafluorophosphate aqueous solution was added dropwise, and a large amount of white solid precipitated. The dropwise addition continued until the white solid completely precipitated. After filtration, the filter cake was dried, and added with an appropriate amount of acetonitrile to prepare a saturated acetonitrile solution. Saturated solution of tetra-n-butylammonium chloride in acetonitrile was added dropwise, and a large amount of white solid precipitated. The dropwise addition continued until the white solid completely precipitated. After filtration, the filter cake was dried to obtain 192 mg of white powdery solid IMC-1, with a yield of 20%. 1H NMR (400 MHz, Deuterium Oxide) δ 8.84 (s, 4H), 7.57 (d, J=1.8 Hz, 4H), 7.44 (s, 8H), 7.39 (d, J=1.7 Hz, 4H), 7.32-7.30 (m, 8H), 7.24-7.22 (m, 8H), 5.42 (s, 8H), 5.33 (s, 8H).

### Example 2

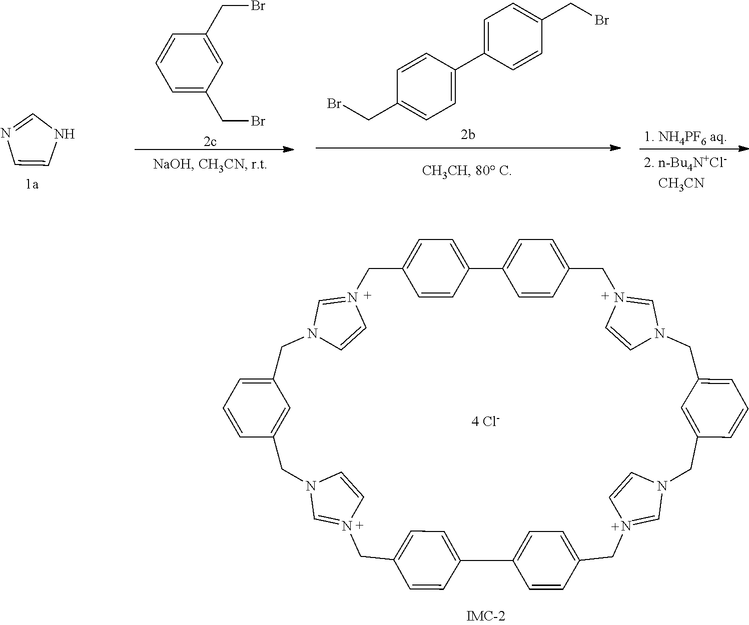

The preparation of IMC-2 first involves the reaction of compound 1a with compound 2c, and then with compound 2b. The specific steps are the same as those for the preparation of IMC-1, with a yield of 19%. 1H NMR (400 MHz, Deuterium Oxide) δ 8.90 (s, 4H), 7.59 (s, 6H), 7.47 (d, J=2.0 Hz, 4H), 7.34-7.32 (m, 12H), 7.26-7.24 (m, 8H), 7.00 (s, 2H), 5.45 (s, 8H), 5.28 (s, 8H).

### Example 3

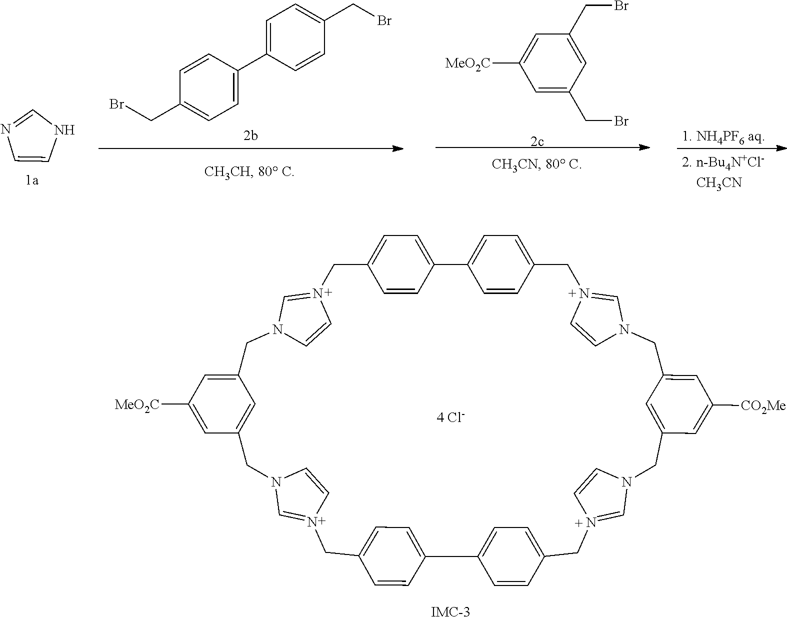

The preparation of IMC-3 first involves the reaction of compound 1a with compound 2b, and then with compound 2d. The specific steps are the same as those for the preparation of IMC-1, with a yield of 13%. 1H NMR (400 MHz, Deuterium Oxide) δ 8.94 (s, 4H), 8.17 (d, J=1.7 Hz, 4H), 7.51 (d, J=2.1 Hz, 4H), 7.38 (d, J=2.0 Hz, 4H), 7.36-7.34 (m, 8H), 7.29-7.28 (m, 10H), 5.53 (s, 8H), 5.31 (s, 8H), 3.92 (s, 6H).

### Example 4

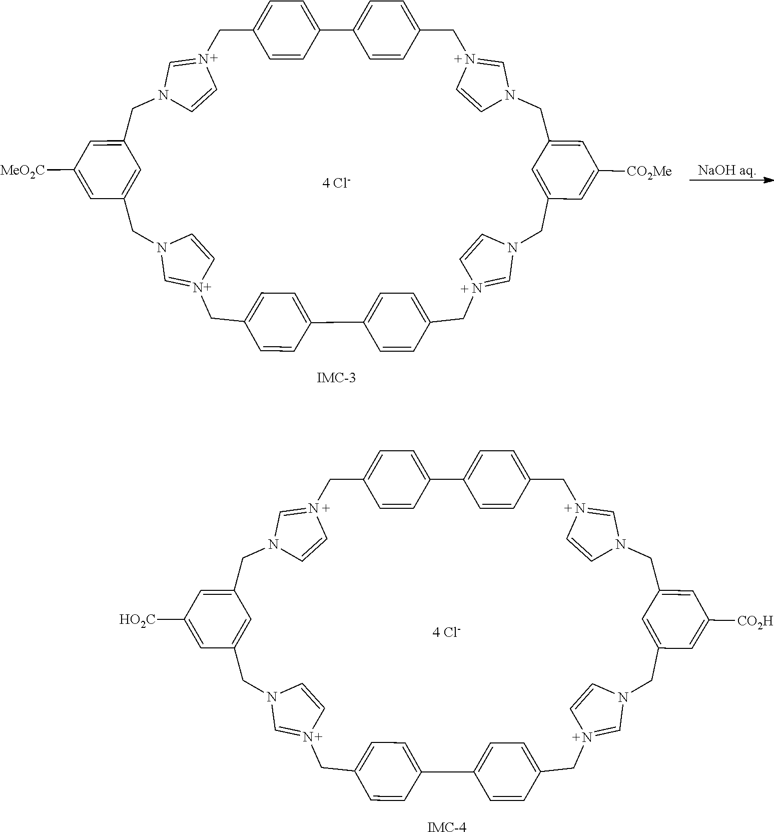

IMC-3 was hydrolyzed to obtain carboxyl substituted IMC-4. The specific steps are as follows: IMC-3 (0.01 g, 0.088 mmol) was dissolved in a mixed solvent of 2 mL of methanol and 2 mL of deionized water. Sodium hydroxide (0.014 g, 0.35 mmol) was added, and the mixture was stirred at room temperature for 12 hours. The reaction solution was spin-dried to remove solvent and added with 5 mL of deionized water to dissolve. 2 M hydrochloric acid solution was added dropwise, and white precipitates were formed. The dropwise addition continued until the solid completely precipitated. After filtration, the filter cake was dried to obtain 79 mg of white solid IMC-4 with a yield of 84%. 1H NMR (400 MHz, Deuterium Oxide) δ 8.92 (s, 4H), 8.16 (s, 4H), 7.48 (s, 4H), 7.37-7.34 (m, 12H), 7.27-7.23 (m, 10H), 5.51 (s, 8H), 5.29 (s, 8H).

### Example 5

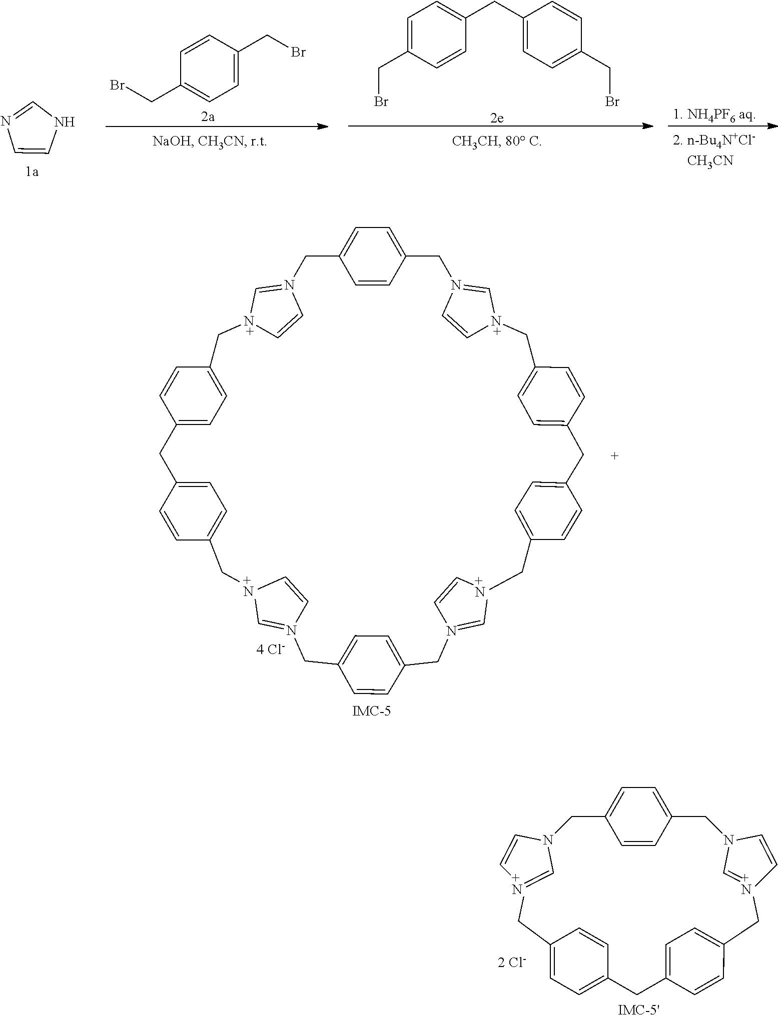

Similar to the preparation of IMC-1, compound 1a reacted with compound 2a, and then reacted with compound 2e to separate and obtain the tetravalent imidazolium salt macrocyclic compound IMC-5 (yield 6%) and the divalent diimidazolium salt cyclic compound IMC-5′ (yield 8%). IMC-5: 1H NMR (400 MHz, Deuterium Oxide) δ 8.71 (s, 4H), 7.51 (d, J=2.0 Hz, 4H), 7.45 (d, J=2.0 Hz, 4H), 7.37 (s, 8H), 7.11 (ABq, J=8.0 Hz, 16H), 5.37 (s, 8H), 5.30 (s, 8H), 3.76 (s, 4H). IMC-5′: 1H NMR (400 MHz, Deuterium Oxide) δ 7.67 (s, 2H), 7.58 (s, 2H), 7.33-7.28 (m, 8H), 7.18 (s, 4H), 5.33 (s, 4H), 5.31 (s, 4H), 3.91 (s, 2H).

### Example 6

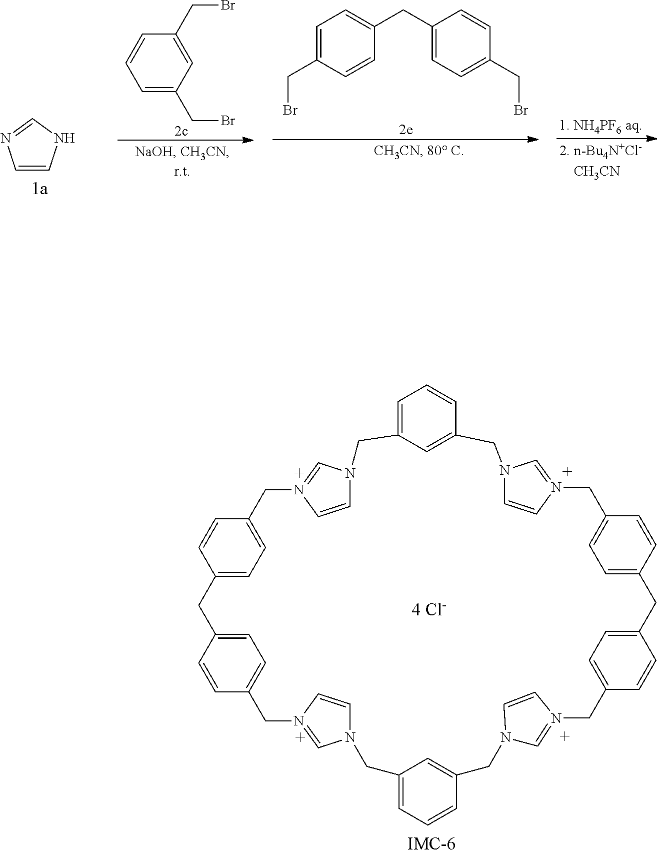

The preparation of IMC-6 first involves the reaction of compound 1a with compound 2c, and then with compound 2e. The specific steps are the same as those for the preparation of IMC-1, with a yield of 26%. 1H NMR (400 MHz, Deuterium Oxide) δ 8.29 (s, 4H), 7.68 (d, J=2.0 Hz, 4H), 7.57 (d, J=2.1 Hz, 4H), 7.47-7.43 (m, 10H), 7.38-7.35 (m, 12H), 7.11 (s, 2H), 5.30 (s, 8H), 5.26 (s, 8H), 3.99 (s, 4H).

### Example 7

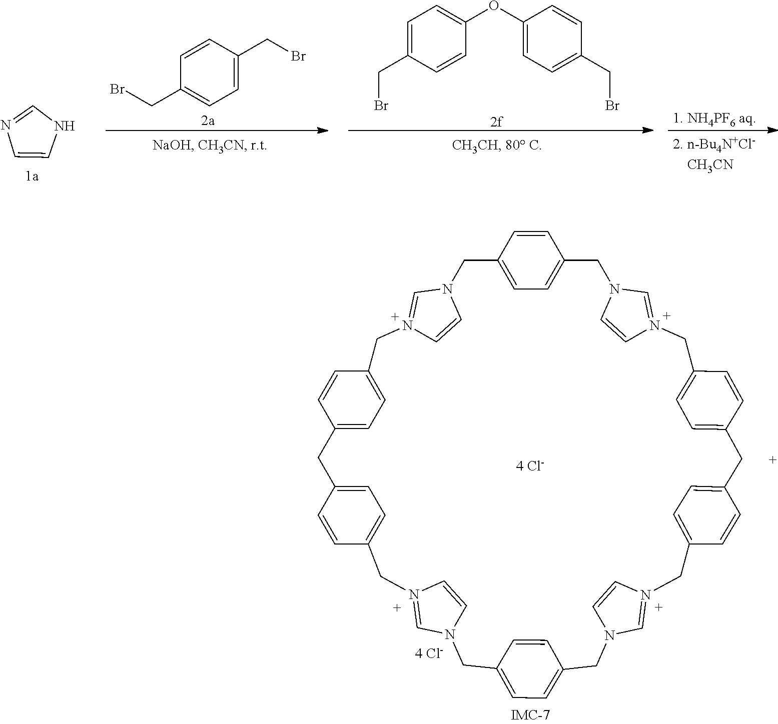

The preparation of IMC-7 first involves the reaction of compound 1a with compound 2a, and then with compound 2f. The specific steps are the same as those for the preparation of IMC-1, with a yield of 7%. 1H NMR (400 MHz, Deuterium Oxide) δ 8.80 (s, 4H), 7.56 (d, J=1.8 Hz, 4H), 7.50 (d, J=1.6 Hz, 4H), 7.44 (s, 8H), 7.32-7.30 (m, 8H), 6.95-6.93 (m, 8H), 5.43 (s, 8H), 5.36 (s, 8H).

### Example 8

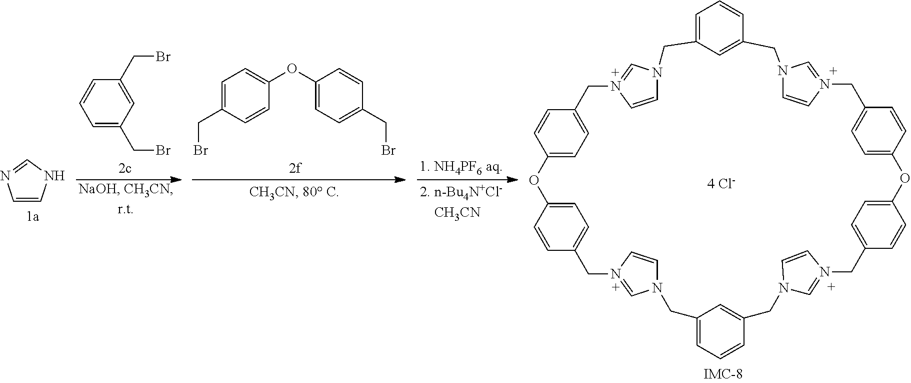

The preparation of IMC-8 first involves the reaction of compound 1a with compound 2c, and then with compound 2f. The specific steps are the same as those for the preparation of IMC-1, with a yield of 16%. 1H NMR (400 MHz, Deuterium Oxide) δ 8.81 (s, 4H), 7.59-7.52 (m, 6H), 7.41 (d, J=1.9 Hz, 4H), 7.39 (d, J=1.9 Hz, 4H), 7.28-7.26 (m, 8H), 7.06 (s, 2H), [symbol] 6.87-6.84 (m, 8H), 5.40 (s, 8H), 5.32 (s, 8H).

### Example 9

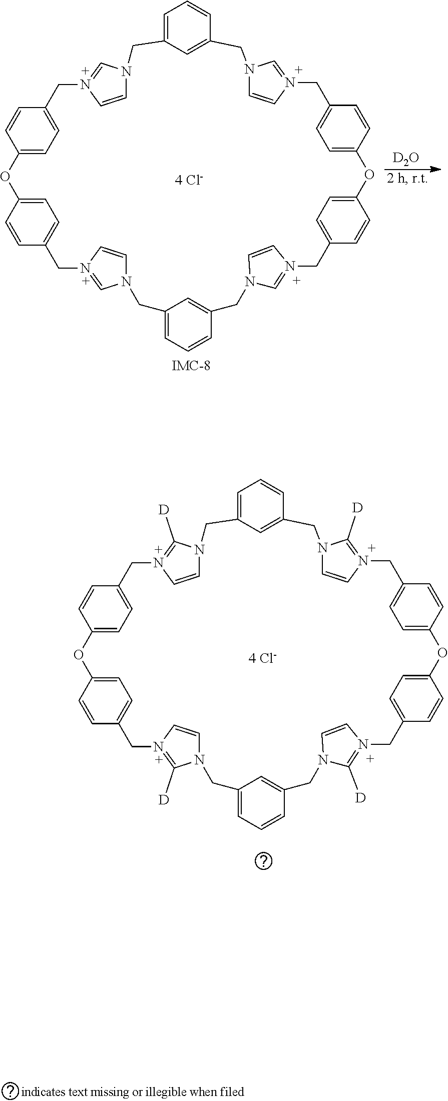

IMC-8 was placed in heavy water and let it stand at room temperature for 2 hours to obtain the deuterated product IMC-8-d₄ with a yield of 100%. 1H NMR (400 MHz, Deuterium Oxide) δ 7.59-7.52 (m, 6H), 7.41 (d, J=1.9 Hz, 4H), 7.39 (d, J=1.9 Hz, 4H), 7.28-7.26 (m, 8H), 7.06 (s, 2H), 6.87-6.84 (m, 8H), 5.40 (s, 8H), 5.32 (s, 8H).

### Example 10

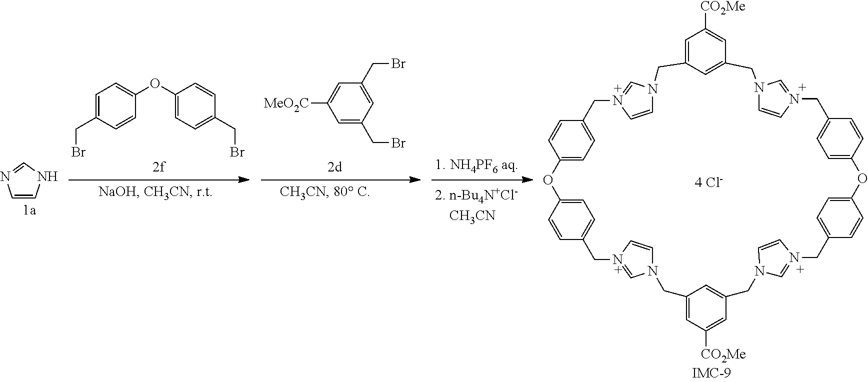

The preparation of IMC-9 first involves the reaction of compound 1a with compound 2f, and then with compound 2d. The specific steps are the same as those for the preparation of IMC-1, with a yield of 18%. 1H NMR (400 MHz, Deuterium Oxide) δ 8.87 (s, 4H), 8.09 (s, 4H), 7.48-7.46 (m, 8H), 7.39 (s, 2H), 7.32-7.30 (m, 8H), 6.89-6.87 (m, 8H), 5.50 (s, 8H), 5.36 (s, 8H), 3.89 (s, 6H).

### Example 11

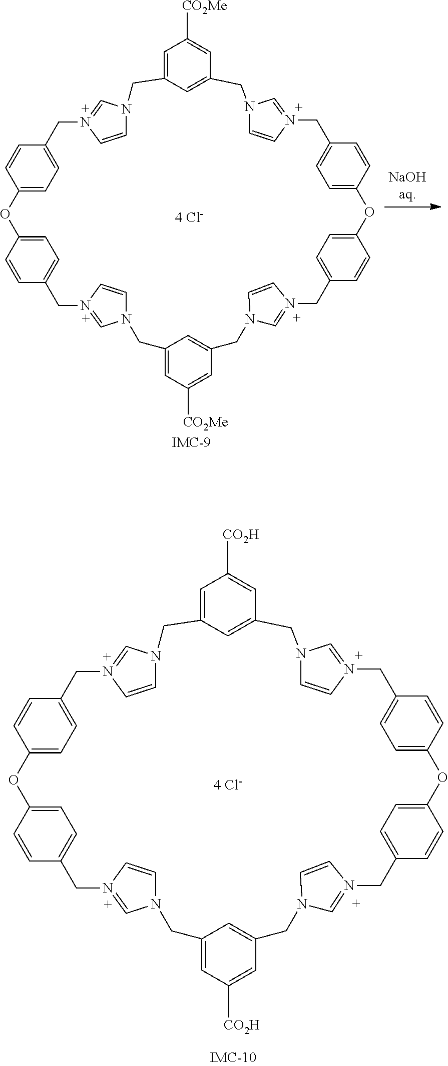

IMC-10 is the hydrolysis product of IMC-9, and the specific steps are the same as the those for the preparation of IMC-4, with a yield of 84%. 1H NMR (400 MHz, Deuterium Oxide) δ 8.83 (s, 4H), 7.94 (d, J=1.6 Hz, 4H), 7.46-7.44 (m, 8H), 7.30-7.28 (m, 8H), 7.26 (s, 2H), 6.86-6.84 (m, 8H), 5.46 (s, 8H), 5.34 (s, 8H).

### Example 12

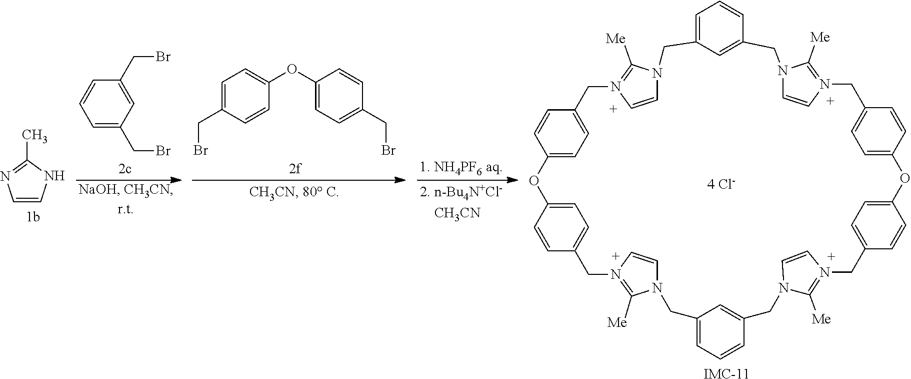

The preparation of IMC-11 first involves the reaction of compound 1b with compound 2c, and then with compound 2f. The specific steps are the same as those for the preparation of IMC-1, with a yield of 13%. 1H NMR (400 MHz, Deuterium Oxide) δ 7.52-7.48 (m, 2H), 7.42-7.40 (m, 4H), 7.33 (s, 4H), 7.24-7.21 (m, 12H), 6.88-6.86 (m, 8H), 6.52 (s, 2H), 5.33 (s, 8H), 5.26 (s, 8H), 2.40 (s, 12H).

### Example 13

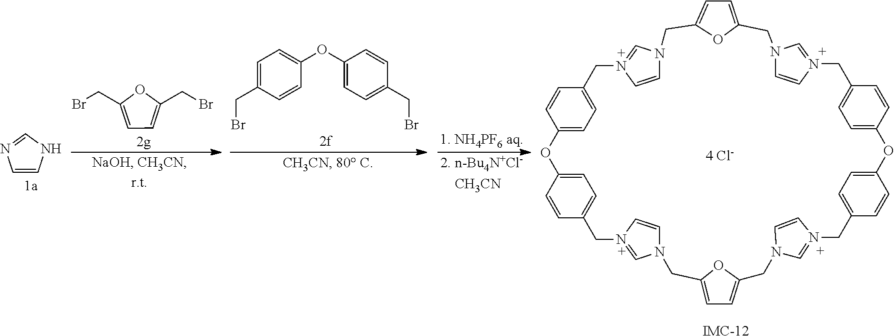

The preparation of IMC-12 first involves the reaction of compound 1a with compound 2g, and then with compound 2f. The specific steps are the same as those for the preparation of IMC-1, with a yield of 16%. 1H NMR (400 MHz, Deuterium Oxide) δ 8.93 (s, 4H), 7.49-7.39 (m, 16H), 7.02 (brs, 8H), 6.65 (s, 4H), 5.40-5.35 (m, 16H).

### Example 14

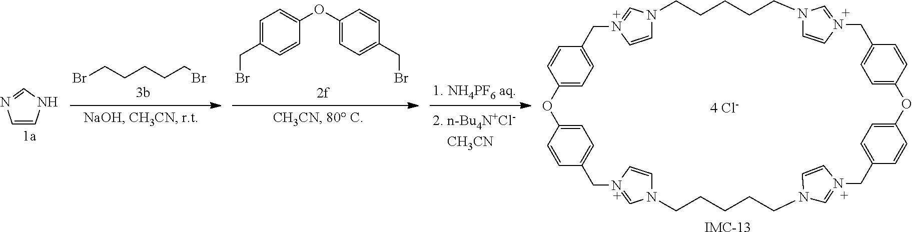

The preparation of IMC-13 first involves the reaction of compound 1a with compound 3b, and then with compound 2f. The specific steps are the same as those for the preparation of IMC-1, with a yield of 14%. 1H NMR (400 MHz, Deuterium Oxide) δ 8.86 (s, 4H), 7.43 (d, J=1.9 Hz, 4H), 7.37 (d, J=1.9 Hz, 4H), 7.36-7.34 (m, 8H), 6.96-6.93 (m, 8H), 5.33 (s, 8H), 4.18 (t, J=6.5 Hz, 8H), 1.88-1.83 (m, 8H), 1.11-1.03 (m, 4H).

### Example 15

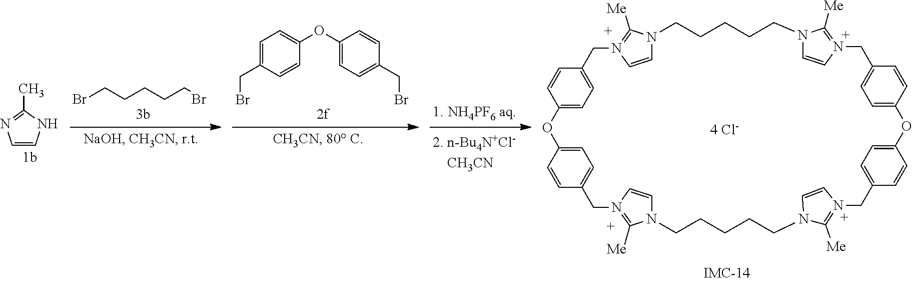

The preparation of IMC-14 first involves the reaction of compound 1b with compound 3b, and then with compound 2f. The specific steps are the same as those for the preparation of IMC-1, with a yield of 13%. 1H NMR (400 MHz, Deuterium Oxide) δ 7.32 (d, J=2.2 Hz, 4H), 7.27-7.25 (m, 12H), 6.96-6.93 (m, 8H), 5.29 (s, 8H), 4.14 (t, J=6.3 Hz, 8H), 2.55 (s, 12H), 1.85-1.78 (m, 8H), 1.10-1.03 (m, 4H).

### Example 16

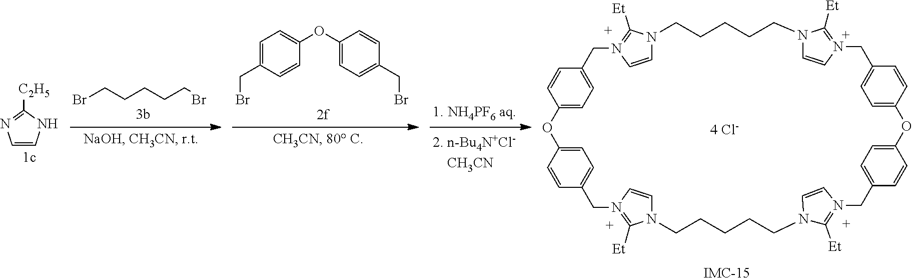

The preparation of IMC-15 first involves the reaction of compound 1c with compound 3b, and then with compound 2f. The specific steps are the same as those for the preparation of IMC-1, with a yield of 15%. 1H NMR (400 MHz, Deuterium Oxide) δ 7.41 (d, J=2.2 Hz, 4H), 7.35 (d, J=2.2 Hz, 4H), 7.26-7.24 (m, 8H), 6.96-6.92 (m, 8H), 5.33 (s, 8H), 4.18 (t, J=6.2 Hz, 8H), 3.01 (q, J=7.6 Hz, 8H), 1.95-1.88 (m, 8H), 1.13-1.05 (m, 4H), 1.01 (t, J=7.6 Hz, 12H).

### Example 17

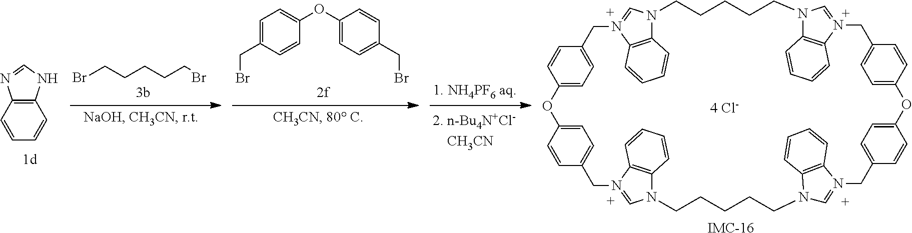

The preparation of IMC-16 first involves the reaction of compound 1d with compound 3b, and then with compound 2f. The specific steps are the same as those for the preparation of IMC-1, with a yield of 14%. 1H NMR (400 MHz, Deuterium Oxide) δ 7.60-7.55 (m, 8H), 7.45-7.36 (m, 8H), 7.00-6.97 (m, 8H), 6.42-6.38 (m, 8H), 5.58 (s, 8H), 4.56 (t, J=6.2 Hz, 8H), 2.11-2.04 (m, 8H), 1.35-1.27 (m, 4H).

### Example 18

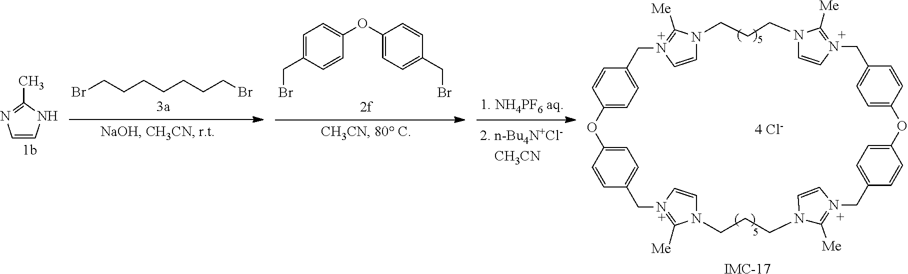

The preparation of IMC-17 first involves the reaction of compound 1b with compound 3a, and then with compound 2f. The specific steps are the same as those for the preparation of IMC-1, with a yield of 14%. 1H NMR (400 MHz, Deuterium Oxide) δ 7.43 (d, J=2.2 Hz, 4H), 7.39 (d, J=2.2 Hz, 4H), 7.26-7.22 (m, 8H), 6.82-6.78 (m, 8H), 5.29 (s, 8H), 4.07 (t, J=6.8 Hz, 8H), 2.56 (s, 12H), 1.65 (t, J=6.8 Hz, 8H), 1.10 (brs, 12H).

### Example 19

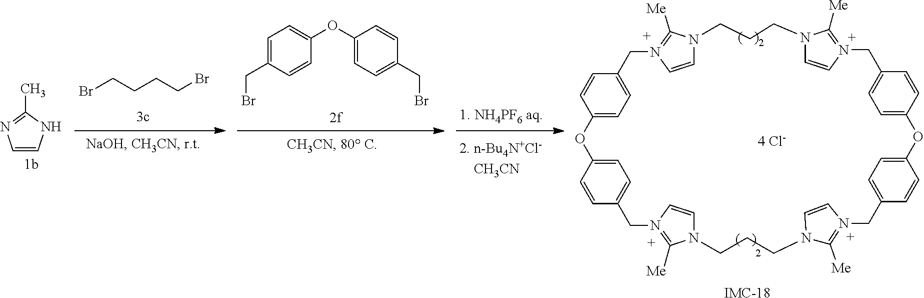

The preparation of IMC-18 first involves the reaction of compound 1b with compound 3c, and then with compound 2f. The specific steps are the same as those for the preparation of IMC-1, with a yield of 14%. JH NMR (400 MHz, Deuterium Oxide) δ 7.37 (d, J=2.2 Hz, 4H), 7.35 (d, J=2.2 Hz, 4H), 7.23-7.19 (m, 8H), 6.89-6.85 (m, 8H), 5.33 (s, 8H), 4.18 (t, J=5.2 Hz, 8H), 2.48 (s, 12H), 1.70-1.66 (m, 8H).

### Example 20

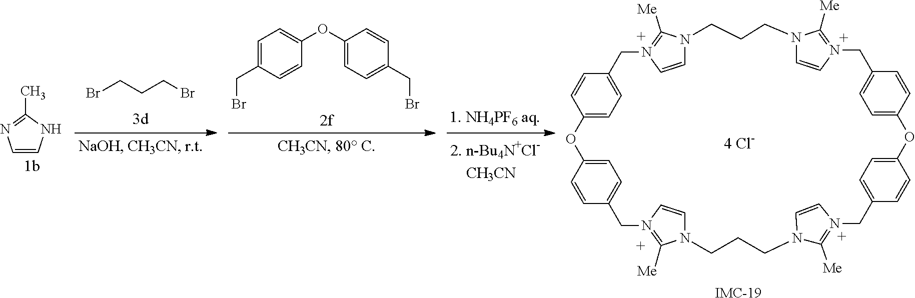

The preparation of IMC-19 first involves the reaction of compound 1b with compound 3d, and then with compound 2f. The specific steps are the same as those for the preparation of IMC-1, with a yield of 16%. 1H NMR (400 MHz, Deuterium Oxide) δ 7.33-7.29 (m, 12H), 7.07-7.03 (m, 12H), 5.26 (s, 8H), 4.33 (t, J=6.3 Hz, 8H), 2.57 (s, 12H), 2.53 (t, J=6.4 Hz, 4H).

### Example 21

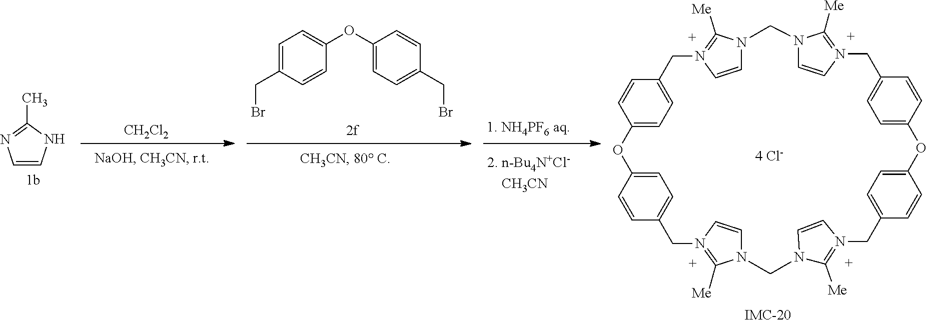

The preparation of IMC-20 first involves the reaction of compound 1b with compound CH₂Cl₂, and then with compound 2f. The specific steps are the same as those for the preparation of IMC-1, with a yield of 14%. 1H NMR (400 MHz, Deuterium Oxide) δ 7.73 (d, J=2.3 Hz, 4H), 7.64 (d, J=2.3 Hz, 4H), 7.26 (d, J=8.3 Hz, 8H), 7.01 (d, J=8.3 Hz, 8H), 6.69 (s, 4H), 5.41 (s, 8H), 2.65 (s, 12H).

### Example 22: Influence of Different Dosages of Imidazolium Salt Macrocyclic Compounds on Neuromuscular Blocking Effect

Taking IMC-19 as an example, SD rats were used as model to test the muscle relaxation effect. Specifically, IMC-19 was formulated as an injection using distilled water as a solvent, with concentrations ranging from 0.8 μg/mL to 16 μg/mL; SD rats weighing 170 to 240 grams were selected as test subjects, and dose groups of 2, 13, 16, 20, and 40 μg/kg were set up, with 2 male and 2 female animals in each group. The following experimental examples were the same.

The operation process is as follows: the rats were anesthetized with isoflurane, at an induction anesthetic dose of 4%, and a maintenance dose of 1%; 10% Na₂S solution was used to remove hair from the right hind leg and nearby area of rats; the airway tube was inserted into the rat trachea and connected to a small animal ventilator with a tidal volume of 1.7 mL and a respiratory rate of 80 breaths per minute; a muscle relaxation monitor was used to test the neuromuscular blockade of the quadriceps femoris muscle in rats. The sensor was fixed to the tibia of the rat, and the electrode was fixed to the right posterior leg of the rat through a patch. Then, the monitor was calibrated using Cal mode until the data became stable, and switched to single stimulation mode (ST). When the ST value became stably ≥90, 10 ST data points were consecutively recorded, and the average values were calculated and recorded as the baseline mean ST₀. According to experimental protocol, IMC-19 injection was administered through the tail vein of rats. The injection volume was adjusted according to the weight of the rats, and the injection operation was completed within 5 seconds. After the injection has completed, start timing and record changes in ST value. When the ST value dropped to the lowest point, 5 ST data points were consecutively recorded, and the average value was calculated and recorded as STₘᵢₙ, (ST₀−STₘᵢₙ)/ST₀ represents the maximum muscle blockade rate of imidazolium salt macrocyclic compounds at that dose.

The experimental results are listed in Table 1. The results showed that there was no blocking effect when the dosage of IMC-19 was 2 μg/kg. As the dosage increased, the maximum muscle blocking rate gradually increased, and when the dosage reached 16 μg/kg, the maximum muscle blocking rate had been over 90%. When the dosage reached 40 μg/kg, the maximum muscle blocking rate reached 100%.

TABLE 1
Dose dependence of Muscle Relaxation Activity of IMC-19

| Dosage (μg/kg) | 2 | 13 | 16 | 20 | 40 |
|---|---|---|---|---|---|
| maximum muscle blockade rate | 0 | 64 | 93 | 99 | 100 |
| $( \frac{{ST}_{0} - {ST}_{min}}{{ST}_{0}} \% )$ | | | | | |

### Example 23: ED95 Test of Muscle Relaxant Activity of Imidazolium Salt Macrocyclic Compounds

Taking IMC-19 as an example, the ED95 for muscle relaxation activity of imidazolium salt macrocyclic compounds was determined using SD rat as model. The specific implementation process was as follows: IMC-19 was prepared as an injection using distilled water as a solvent, with concentrations ranging from 0.8 μg/mL to 16 μg/mL; dose groups of 2 μg/kg, 10 μg/kg, 13 μg/kg, 16 μg/kg, 20 μg/kg, and 40 μg/kg were set up, with 2 female and 2 male animals assigned to each group. The actual injection volume was calculated based on rat body weight and injection concentration. The operation process was the same as in Example 22, and the maximum muscle blockade rate (ST₀−STₘᵢₙ)/ST₀ at each dose was recorded separately. The experimental results were fitted using GraphPad Prism software to obtain the drug inhibition curve, and the ED95 of IMC-19 was calculated to be about 17.5 μg/kg.

### Example 24: Screening Test of Neuromuscular Blocking Effect Under Specific Dosage

The neuromuscular blocking effect of imidazolium salt macrocyclic compounds under specific dosages was screened and tested.

Specifically, imidazolium salt macrocyclic compounds were prepared into injections using distilled water as a solvent, and dosage groups ranging from 20 μg/kg-1800 μg/kg were set up, with 2 male and 2 female animals in each dosage group. The operation process was the same as in Example 22. Start timing and record changes in ST value after the injection is completed: when the ST value decreases to 95% of ST₀, record time t1; when the ST value returns to 25% of ST₀, record time t2; when the ST value returns to 90% of ST₀, record the time t3, t1 represents the onset time of muscle relaxation, t2−t1 represents the duration of muscle relaxation, and t3-t2 represents the recovery time of muscle relaxation; record the maximum muscle blockade rate; all results are reported as the mean value from 4 animals per group.

The experimental results are as shown in Table 2. IMC-1, IMC-5, IMC-7, IMC-8, IMC-10, IMC-1, and IMC-13 can achieve muscle blocking of over 90% at a certain dosage. IMC-14, IMC-15, IMC-17, IMC-18, and IMC-19 can achieve 100% muscle blocking at lower dosages of 20 μg/kg-40 μg/kg. The bis-imidazolium salt macrocyclic compound IMC-5′ exhibited a maximum muscle blockade rate of only 23.3% at a dose of 2000 μg/kg, indicating that the muscle blocking activity of tetravalent imidazolium salt macrocyclic compounds were much higher than that of divalent cyclic compounds.

TABLE 2
The dosage and maximal neuromuscular blocking
rate of imidazolium salt macrocyclic compounds

| | | maximum | | | maximum |
|---|---|---|---|---|---|
| | | muscle | | | muscle |
| | | blockade | | | blockade |
| | Dosage | rate | | Dosage | rate |
| Compound | (μg/kg) | (%) | Compound | (μg/kg) | (%) |
| IMC-1 | 1200 | 97 | IMC-2 | 300 | 43 |
| IMC-5 | 1800 | 100 | IMC-6 | 1200 | 44 |
| IMC-7 | 1200 | 98 | IMC-8 | 160 | 100 |
| IMC-9 | 600 | 100 | IMC-10 | 1000 | 90 |
| IMC-11 | 150 | 100 | IMC-13 | 60 | 95 |
| IMC-14 | 40 | 100 | IMC-15 | 40 | 100 |
| IMC-17 | 20 | 100 | IMC-18 | 40 | 100 |
| IMC-19 | 40 | 100 | IMC-20 | 40 | 63 |
| IMC-5′ | 2000 | 23.3 | | | |

### Example 25: Onset Time and Duration of Imidazolium Salt Macrocyclic Compounds

Using the experimental conditions described in Example 24, monitoring on the onset time and duration of muscle relaxation induced by imidazolium salt macrocyclic compounds with a maximum muscle blockade rate of 100% was further conducted. The results are listed in the Table 3. The experimental results showed that imidazolium salt macrocyclic compounds at high doses can achieve rapid onset (0.24 min) and long-acting muscle relaxation (5 hours).

TABLE 3
The onset time and duration of muscle relaxation under different
doses of imidazolium salt macrocyclic compounds

| | Dosage | | | | |
|---|---|---|---|---|---|
| | (μg/kg) | Onset time | | Duration | |
| IMC-8 | 160 | 3 | min | 5 | min |
| IMC-13 | 300 | 0.35 | min | 3 | h |
| IMC-14 | 40 | 2 | min | 2 | h |
| IMC-14 | 100 | 0.47 | min | 4 | h |
| IMC-15 | 40 | 3 | min | 2 | h |
| IMC-17 | 40 | 3 | min | 2 | h |
| IMC-18 | 40 | 2 | min | 2 | h |
| IMC-19 | 40 | 2 | min | 2 | h |
| IMC-19 | 60 | 1.3 | min | 3 | h |
| IMC-19 | 200 | 0.35 | min | 4 | h |
| IMC-19 | 600 | 0.24 | min | 5 | h |

### Example 26: Antagonistic Test of Imidazolium Salt Macrocyclic Compounds

Taking IMC-19 as an example, the antagonistic effect of cholinesterase inhibitors on imidazolium salt macrocyclic compounds was detected using Neostigmine as an antagonist.

Specifically, imidazolium salt macrocyclic compounds were prepared into injections using distilled water as a solvent, with a concentration of 8.0 μg/mL; Neostigmine was formulated into an injection using distilled water as a solvent, with a concentration of 48 μg/mL; the dosage of IMC-19 was 20 μg/kg, and the dosage of antagonist was 240 μg/kg. The actual injection volume was calculated based on the body weight of the rats and the injection concentration, with 2 female and 2 male animals per group, and the results were reported as the mean value. The operation process was the same as in Example 22, starting timing and recording changes in ST value after injection was completed. When the ST value dropped to the lowest point and the monitoring data stabilized, the rats were injected with Neostigmine solution through the tail vein. After the injection was completed, the time and changes in ST value were recorded.

The experimental results indicated that Neostigmine can rapidly antagonize muscle blockade caused by imidazolium salt macrocyclic compounds. Due to the fact that Neostigmine is a cholinesterase inhibitor, which inhibits the hydrolysis of acetylcholine in the body, thereby achieving the enrichment of acetylcholine and competitively squeezing out the imidazolium salt macrocycle on the acetylcholine receptor, thereby restoring the transmission of neuromuscular excitation and ultimately achieving the reversal effect of muscle relaxation.

The experimental results indicate that imidazolium salt macrocyclic compounds are a class of competitive N2 cholinergic receptor inhibitors, which conform to the characteristics of non-depolarizing muscle relaxants and indicate their high safety.

All documents mentioned in the present invention are cited as references in this application, just as each document is individually cited as a reference. In addition, it should be understood that, after reading the above teaching content of the present invention, those skilled in the art can make various changes or modifications to the present invention, and these equivalent forms also fall within the scope defined by the appended claims of the present application.

1. An imidazolium salt macrocyclic compound of formula I, or a pharmaceutically acceptable salt, a hydrate, or a solvate thereof, characterized in that, 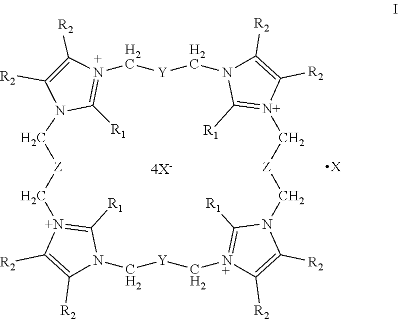
each R₁ is independently selected from the group consisting of: H, D, substituted or unsubstituted C₁₋₁₀ alkyl, substituted or unsubstituted 1-10 membered heteroalkyl containing 1 to 6 heteroatoms selected from nitrogen, oxygen, and sulfur, substituted or unsubstituted C₂₋₁₀ alkenyl, substituted or unsubstituted 2-10 membered heteroalkenyl containing 1 to 3 heteroatoms selected from nitrogen, oxygen, and sulfur, substituted or unsubstituted C₂₋₁₀ alkynyl, substituted or unsubstituted 2-10 membered heteroalkynyl containing 1 to 3 heteroatoms selected from nitrogen, oxygen, and sulfur, substituted or unsubstituted C₃₋₁₀ cycloalkyl, substituted or unsubstituted 3-10 membered heterocyclyl containing 1 to 6 heteroatoms selected from nitrogen, oxygen, and sulfur;
each R² is independently selected from the group consisting of: H, substituted or unsubstituted C₁₋₁₀ alkyl, substituted or unsubstituted 1-10 membered heteroalkyl containing 1 to 6 heteroatoms selected from nitrogen, oxygen, and sulfur, substituted or unsubstituted C₂₋₁₀ alkenyl, substituted or unsubstituted 2-10 membered heteroalkenyl containing 1 to 3 heteroatoms selected from nitrogen, oxygen, and sulfur, substituted or unsubstituted C₂₋₁₀ alkynyl, substituted or unsubstituted 2-10 membered heteroalkynyl containing 1 to 3 heteroatoms selected from nitrogen, oxygen, and sulfur, or two adjacent R₂ together with their attached carbon atoms form a group selected from the group consisting of: substituted or unsubstituted C₃₋₁₀ cycloalkyl, substituted or unsubstituted C₆₋₁₀ aryl, substituted or unsubstituted 3-10 membered heterocyclyl containing 1 to 3 heteroatoms selected from nitrogen, oxygen, and sulfur, and substituted or unsubstituted 5-10 membered heteroaryl containing 1 to 6 heteroatoms selected from nitrogen, oxygen, and sulfur;
each Y is independently selected from the group consisting of: null, substituted or unsubstituted C₁₋₁₀ alkylene, substituted or unsubstituted 1-10 membered heteroalkylene containing 1 to 3 heteroatoms selected from nitrogen, oxygen, and sulfur, substituted or unsubstituted C₃₋₁₀ cycloalkylene, substituted or unsubstituted C₅₋₁₀ arylene, substituted or unsubstituted Ar1-L-Ar2, and substituted or unsubstituted 5-10 membered heteroarylene;
each Z is independently selected from the group consisting of: substituted or unsubstituted Ar1-L-Ar2, and substituted or unsubstituted 5-10 membered heteroarylene;
L is selected from the group consisting of: null, O, NH, C₁₋₆ alkylene, S, and phenylene;
Ar1 and Ar2 are each independently null, phenylene, or 5 membered heteroarylene;
X is a pharmaceutically acceptable anion charge-balanced with the tetravalent cationic imidazolium macrocycle in the structure of formula I;
unless otherwise specified, the “substituted” refers to the substitution of one or more hydrogens on a group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

2. The compound according to claim 1, characterized in that each R₁ is independently selected from the group consisting of: H, D, substituted or unsubstituted C₁₋₆ alkyl, substituted or unsubstituted 1-6 membered heteroalkyl containing 1 to 3 heteroatoms selected from nitrogen, oxygen, and sulfur, substituted or unsubstituted C₂₋₆ alkenyl, substituted or unsubstituted 2-6 membered heteroalkenyl containing 1 to 3 heteroatoms selected from nitrogen, oxygen, and sulfur, substituted or unsubstituted C₂₋₆ alkynyl, substituted or unsubstituted 2-6 membered heteroalkynyl containing 1 to 3 heteroatoms selected from nitrogen, oxygen, and sulfur, substituted or unsubstituted C₃₋₇ cycloalkyl, and substituted or unsubstituted 3-7 membered heterocyclyl containing 1 to 3 heteroatoms selected from nitrogen, oxygen, and sulfur;
each R₂ is independently selected from the group consisting of: each R₂ is independently selected from the group consisting of: H, substituted or unsubstituted C₁₋₆ alkyl, substituted or unsubstituted 1-6 membered heteroalkyl containing 1 to 3 heteroatoms selected from nitrogen, oxygen, and sulfur, substituted or unsubstituted C₂₋₆ alkenyl, substituted or unsubstituted 2-6 membered heteroalkenyl containing 1 to 3 heteroatoms selected from nitrogen, oxygen, and sulfur, substituted or unsubstituted C₂₋₆ alkynyl, substituted or unsubstituted 2-6 membered heteroalkynyl containing 1 to 3 heteroatoms selected from nitrogen, oxygen, and sulfur, or two adjacent R₂ together with their attached carbon atoms form a group selected from the group consisting of: substituted or unsubstituted C₃₋₇ cycloalkyl, substituted or unsubstituted C₆₋₁₀ aryl, substituted or unsubstituted 3-7 membered heterocyclyl containing 1 to 3 heteroatoms selected from nitrogen, oxygen, and sulfur, and substituted or unsubstituted 5-7 membered heteroaryl containing 1 to 3 heteroatoms selected from nitrogen, oxygen, and sulfur;
each Y is independently selected from the group consisting of: null, substituted or unsubstituted C₁₋₁₀ alkylene, substituted or unsubstituted 1-10 membered heteroalkylene containing 1 to 3 heteroatoms selected from nitrogen, oxygen, and sulfur, substituted or unsubstituted C₃₋₁₀ cycloalkylene, substituted or unsubstituted C₅₋₁₀ arylene, substituted or unsubstituted Ar1-L-Ar2, and substituted or unsubstituted 5-10 membered heteroaryl;
each Z is independently selected from the group consisting of: substituted or unsubstituted Ar1-L-Ar2, and substituted or unsubstituted 5-membered heteroaryl;
L is selected from the group consisting of: null, O, S, NH, C₁₋₆ alkylene, and phenylene;
Ar1 and Ar2 are each independently null, phenylene, or 5-membered heteroarylene.

3. The compound according to claim 1, characterized in that each R₁ is independently selected from the group consisting of: H, D, substituted or unsubstituted C₁₋₆ alkyl, substituted or unsubstituted 1-6 membered heteroalkyl containing 1 to 3 heteroatoms selected from nitrogen, oxygen, and sulfur, substituted or unsubstituted C₂₋₆alkenyl, and substituted or unsubstituted C₃₋₇ cycloalkyl;
each R₂ is independently selected from the group consisting of: H, substituted or unsubstituted C₁₋₆ alkyl, substituted or unsubstituted 1-6 membered heteroalkyl containing 1 to 3 heteroatoms selected from nitrogen, oxygen, and sulfur, or two adjacent R₂ together with their attached carbon atoms form a group selected from the group consisting of: substituted or unsubstituted C₃₋₇ cycloalkyl, substituted or unsubstituted phenyl;
each Y is independently null, substituted or unsubstituted C₁₋₇ alkylene, substituted or unsubstituted 1-7 membered heteroalkylene containing 1 to 3 heteroatoms selected from nitrogen, oxygen, and sulfur, substituted or unsubstituted C₃₋₇ cycloalkylene, substituted or unsubstituted phenyl, substituted or unsubstituted Ar1-L-Ar2, and substituted or unsubstituted 5-8 membered heteroaryl;
each Z is independently substituted or unsubstituted Ar1-L-Ar2, and substituted or unsubstituted 5-8 membered heteroaryl;
L is selected from the group consisting of: null, O, S, NH, C₁₋₆ alkylene, and phenylene;
Ar1 and Ar2 are each independently null, phenylene, or 5-membered heteroarylene.

4. The compound according to claim 1, characterized in that R₁ is H, D, substituted or unsubstituted C₁₋₃ alkyl, and allyl; wherein, the “substituted” refers to the substitution of hydrogen atoms on the group by one or more C₁₋₄ alkyl or halogen.

5. The compound according to claim 1, characterized in that 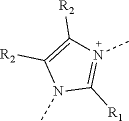
is selected from 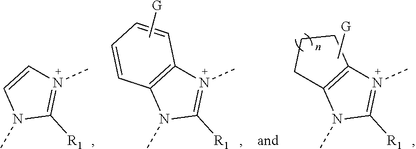
wherein, n=1, 2; G is H, or one or more groups selected from the group consisting of: C₁₋₆ alkyl, C₁₋₆ alkoxy, and C₁₋₆ alkoxycarbonyl.

6. The compound according to claim 1, characterized in that each Y is independently null, or a group that is substituted or unsubstituted and is selected from the group consisting of: 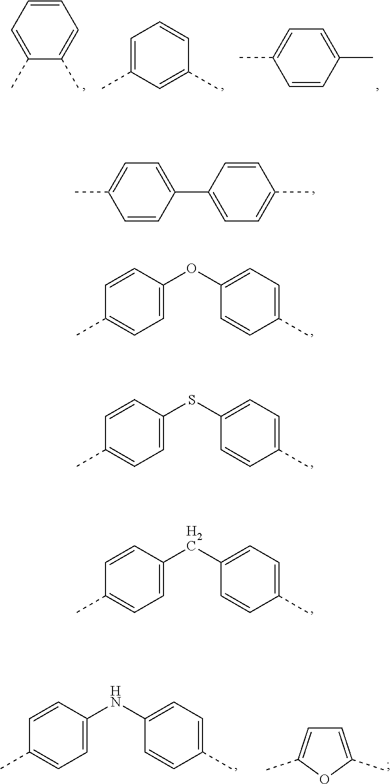
each Z is independently a group that is substituted or unsubstituted and is selected from the group consisting of: 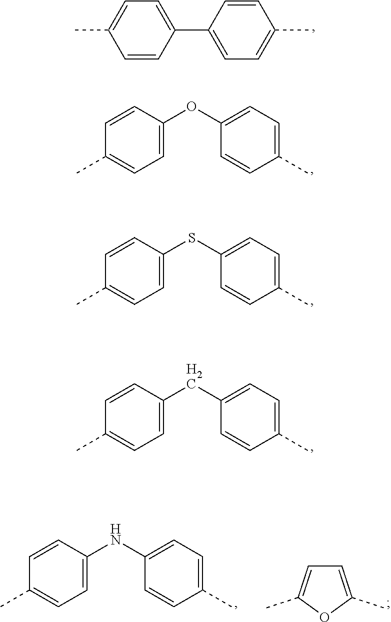
wherein, the “substituted” refers to the substitution of one or more hydrogen atoms on the group by a group selected from the group consisting of: C₁₋₆ alkyl, halogen, C₁₋₆ alkoxycarbonyl, and amido.

7. The compound according to claim 1, characterized in that each R₂ is independently hydrogen, or two adjacent R₂ together with their attached carbon atoms form a substituted or unsubstituted benzene ring, cyclopentyl, or cyclohexyl.

8. The compound of claim 1, characterized in that the tetravalent cationic imidazolium macrocycle in the compound is selected from group consisting of: 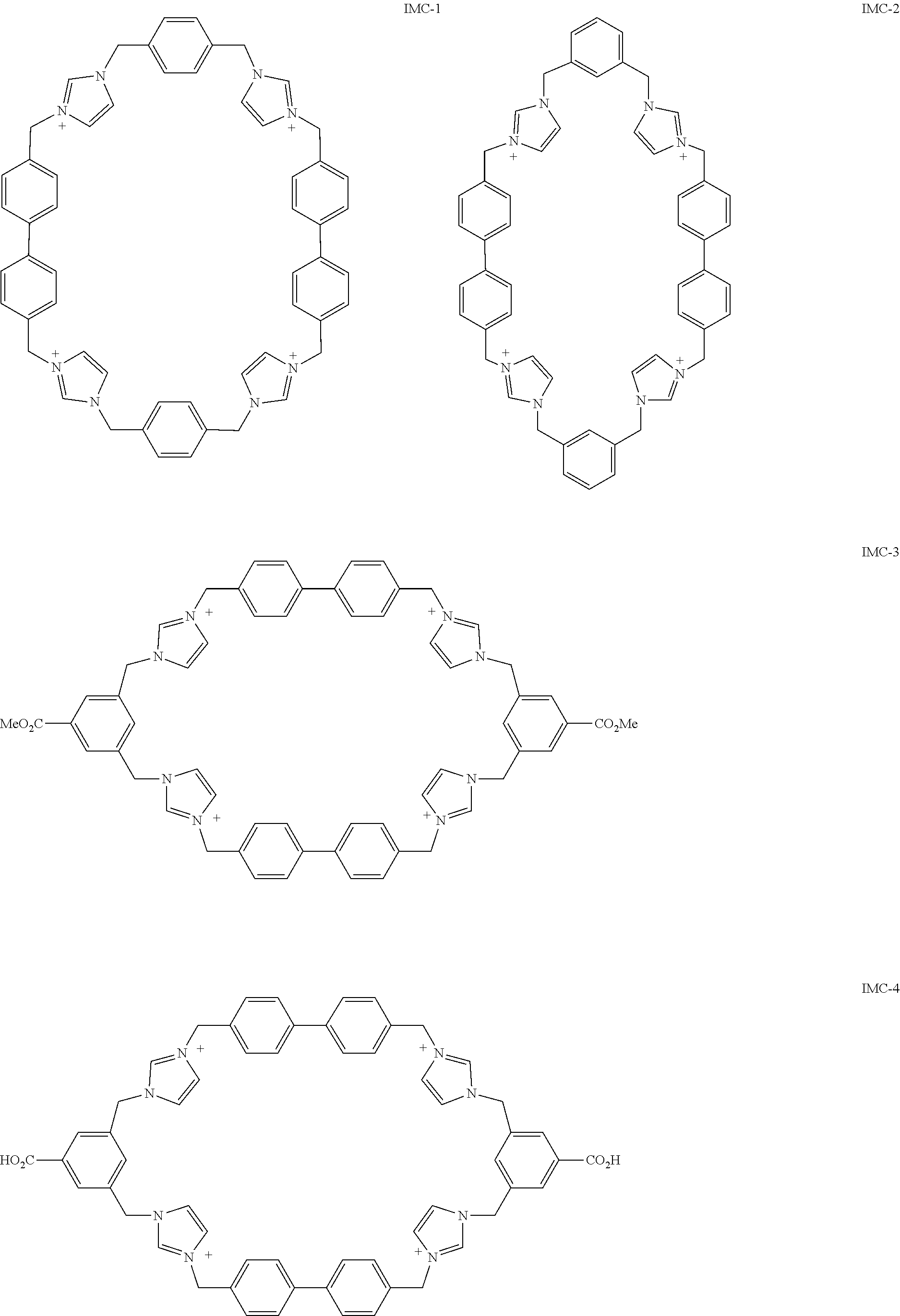 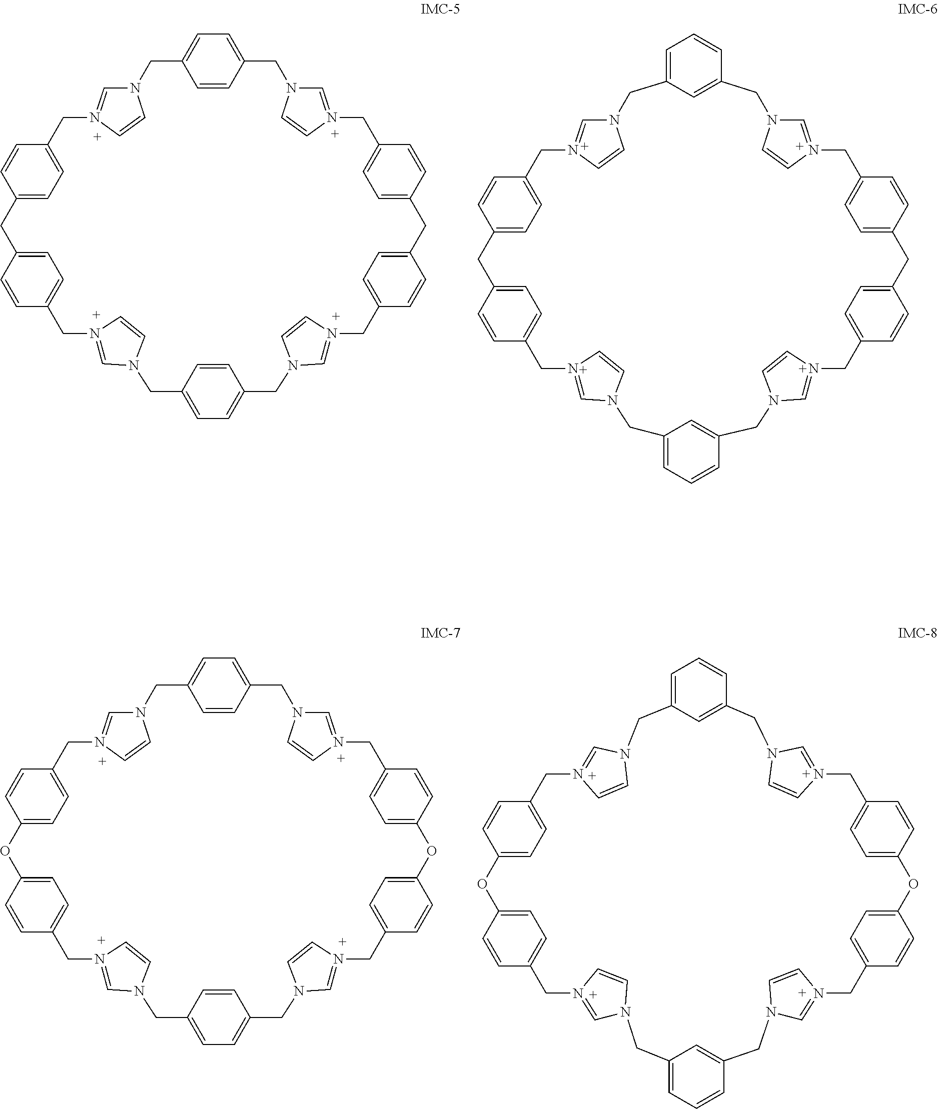 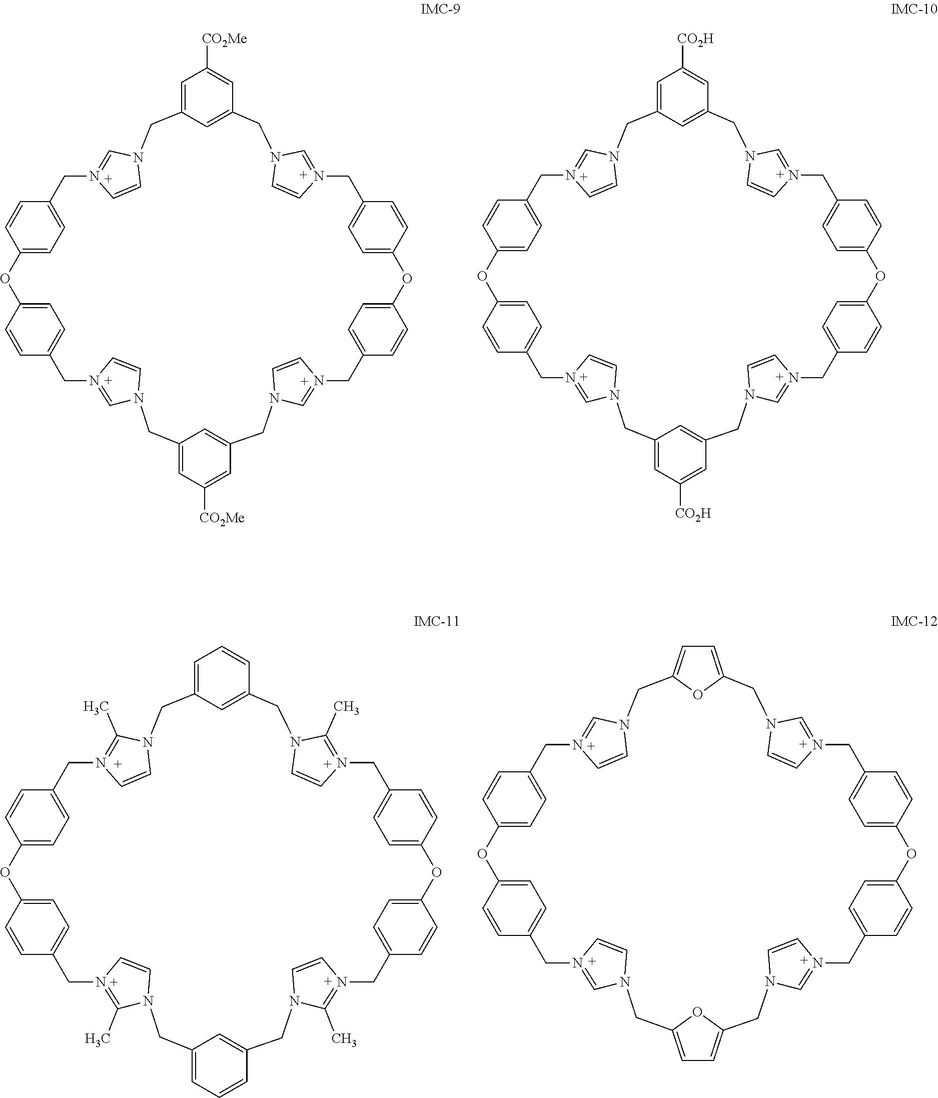 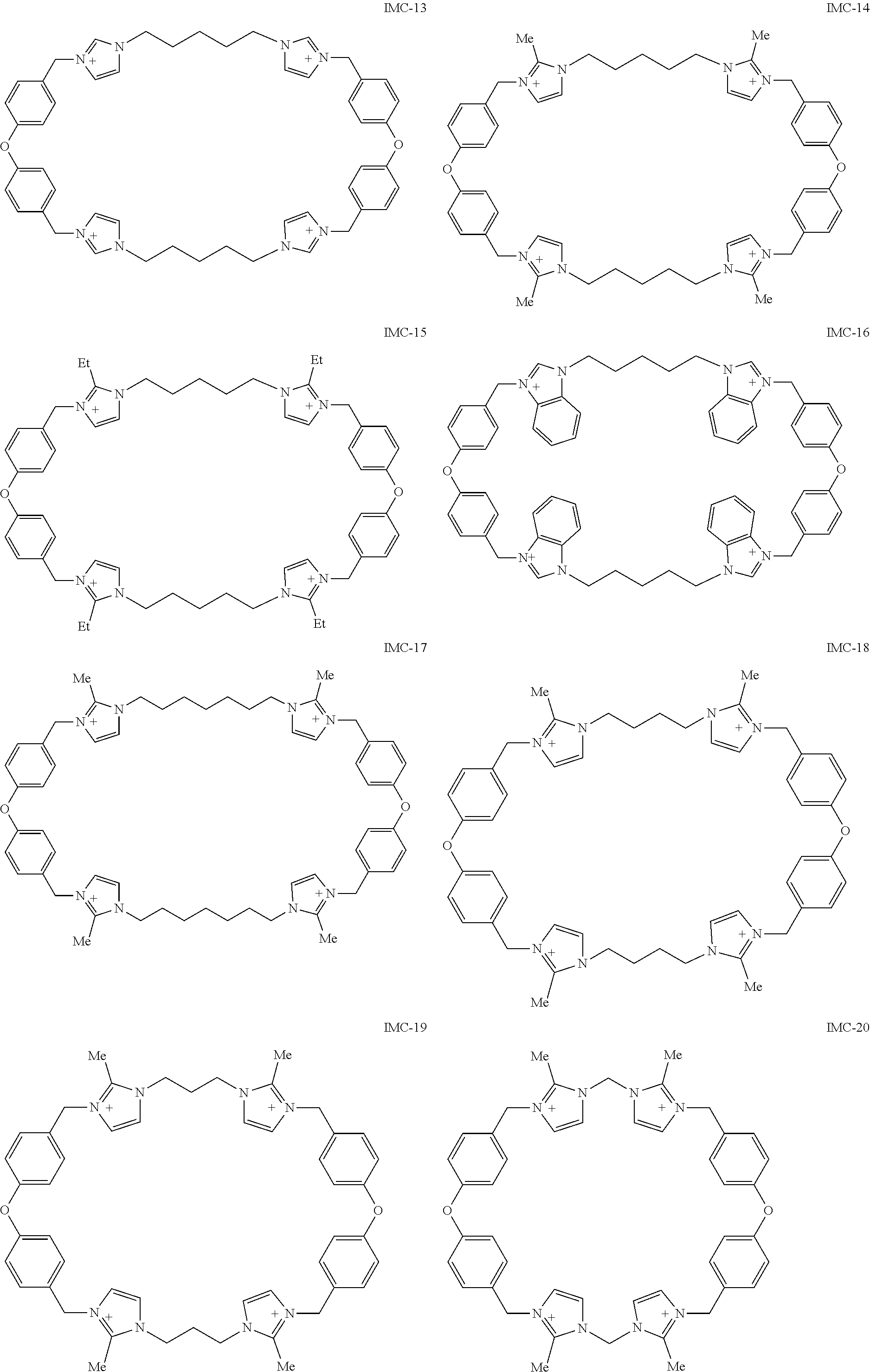
wherein, X is an anion paired with the tetravalent cationic imidazolium macrocycle; and the anion is independently selected from the group consisting of: Cl⁻, Br⁻, I⁻, SO₄²⁻, HSO₄⁻, PO₄³⁻, HOPO₃²⁻, (HO)₂PO₂⁻, R′CO₂⁻, ⁻O₂C(CH₂)ₙCO₂⁻ (n=0-20), ArCO₂⁻, R′SO₃⁻, ArSO₃⁻, R′PO₃⁻, ArPO₃⁻, alginate, citrate, aspartate, camphorate, camphorsulfonate, digluconate, glycerophosphate, fumarate, taurate, 2-hydroxyethanesulfonate, lactate, maleate, nicotinate, pamoate, pectate, thiocyanate, 3-phenylpropionate, picrate, and tartrate; wherein, R′ is substituted or unsubstituted C₁₋₁₀ alkyl, and Ar is substituted or unsubstituted C₅₋₇ aryl, or substituted or unsubstituted 5-7 membered heteroaryl containing 1-3 heteroatoms selected from N, O, and S,
wherein, the “substituted” refers to the substitution of one or more hydrogen atoms on the group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.

9. A pharmaceutical composition, characterized in that comprising
(a) the compound according to claim 1, or the pharmaceutically acceptable salt, the hydrate or the solvate thereof as active ingredient; and (b) pharmaceutically acceptable carriers.

10. A pharmaceutical composition, characterized in that comprising:
(a) The imidazolium salt macrocyclic compound according to claim 1 as a first active ingredient;
(b) An ingredient having muscle relaxation effects as a second active ingredient that is selected from the group consisting of: rocuronium bromide, vecuronium bromide, cisatracurium besylate, pancuronium bromide, or a combination thereof; and
(c) pharmaceutically acceptable carriers or excipients.

11-13. (canceled)

14. A method for relaxing skeletal muscles, comprising steps of: administering to a subject in need thereof a therapeutically effective amount of the imidazolium salt macrocyclic compound of formula I, or a pharmaceutically acceptable salt, a hydrate, or a solvate thereof, or the pharmaceutical composition comprising the imidazolium salt macrocyclic compound of formula I, or a pharmaceutically acceptable salt, a hydrate, or a solvate thereof: 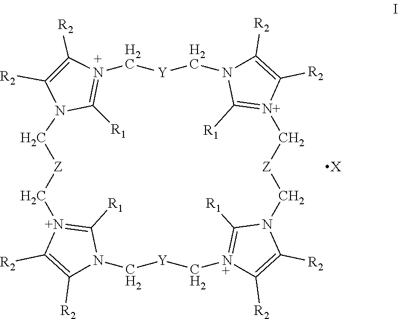
each R₁ is independently selected from the group consisting of: H, D, substituted or unsubstituted C₁₋₁₀ alkyl, substituted or unsubstituted 1-10 membered heteroalkyl containing 1 to 6 heteroatoms selected from nitrogen, oxygen, and sulfur, substituted or unsubstituted C₂₋₁₀ alkenyl, substituted or unsubstituted 2-10 membered heteroalkenyl containing 1 to 3 heteroatoms selected from nitrogen, oxygen, and sulfur, substituted or unsubstituted C₂₋₁₀ alkynyl, substituted or unsubstituted 2-10 membered heteroalkynyl containing 1 to 3 heteroatoms selected from nitrogen, oxygen, and sulfur, substituted or unsubstituted C₃₋₁₀ cycloalkyl, and substituted or unsubstituted 3-10 membered heterocyclyl containing 1 to 6 heteroatoms selected from nitrogen, oxygen, and sulfur;
each R₂ is independently selected from the group consisting of: H, substituted or unsubstituted C₁₋₁₀ alkyl, substituted or unsubstituted 1-10 membered heteroalkyl containing 1 to 6 heteroatoms selected from nitrogen, oxygen, and sulfur, substituted or unsubstituted C₂₋₁₀ alkenyl, substituted or unsubstituted 2-10 membered heteroalkenyl containing 1 to 3 heteroatoms selected from nitrogen, oxygen, and sulfur, substituted or unsubstituted C₂₋₁₀ alkynyl, substituted or unsubstituted 2-10 membered heteroalkynyl containing 1 to 3 heteroatoms selected from nitrogen, oxygen, and sulfur, or two adjacent R₂ together with their attached carbon atoms form a group selected from the group consisting of: substituted or unsubstituted C₃₋₁₀ cycloalkyl, substituted or unsubstituted C₆₋₁₀ aryl, substituted or unsubstituted 3-10 membered heterocyclyl containing 1 to 3 heteroatoms selected from nitrogen, oxygen, and sulfur, and substituted or unsubstituted 5-10 membered heteroaryl containing 1 to 6 heteroatoms selected from nitrogen, oxygen, and sulfur;
each Y and Z are independently selected from the group consisting of: null, substituted or unsubstituted C₁₋₁₀ alkylene, substituted or unsubstituted 1-10 membered heteroalkylene containing 1 to 3 heteroatoms selected from nitrogen, oxygen, and sulfur, substituted or unsubstituted C₃₋₁₀ cycloalkylene, substituted or unsubstituted C₅₋₁₀ arylene, substituted or unsubstituted Ar1-L-Ar2, and substituted or unsubstituted 5-10 membered heteroarylene;
L is selected from the group consisting of: null, O, S, NH, C₁₋₆ alkylene, and phenylene;
Ar1 and Ar2 are each independently null, phenylene, or 5-membered heteroarylene;
X is a pharmaceutically acceptable anion charge-balanced with the tetravalent cationic imidazolium macrocycle in the structure of formula I;
unless otherwise specified, the “substituted” refers to the substitution of one or more hydrogens on a group by a group selected from the group consisting of: halogen, nitro, amino, cyano, hydroxyl, amido, trifluoromethyl, C₁₋₆alkyl, C₂₋₆alkenyl, C₂₋₆alkynyl, C₁₋₆alkoxy, phenyl, benzyl, 3-6 membered heterocyclyl, and C₁₋₆alkoxycarbonyl.
